(19) United States
(12) Patent Application Publication
(10) Pub. No.: US 2017/0081734 A1
(43) Pub. Date: Mar. 23, 2017

(54) WHEAT WITH ELEVATED FRUCTAN, ARABINOXYLAN

(71) Applicants: Dow AgroSciences LLC, Indianapolis, IN (US); CSIRO, Acton (AU)

(72) Inventors: Stephen Alan Jobling, Nicholls (AU); Xinguo Li, Duffy (AU); Klara Louise Verbyla, Isaacs (AU); Colin Robert Cavanagh, Bardon (AU)

(73) Assignees: Dow AgroSciences LLC, Indianapolis, IN (US); CSIRO, Acton (AU)

(21) Appl. No.: 15/270,043

(22) Filed: Sep. 20, 2016

Related U.S. Application Data
(60) Provisional application No. 62/377,763, filed on Aug. 22, 2016.
(60) Provisional application No. 62/221,348, filed on Sep. 21, 2015.

(51) Int. Cl. C12Q 1/68; A01H 5/10

(52) U.S. Cl. CPC C12Q 1/6895; A01H 5/10; C12Q 2600/13; C12Q 2600/156

(57) ABSTRACT

This invention relates to methods for identifying wheat plants that having increased fructan/arabinoxylan. The methods use molecular markers to identify and to select plants with increased fructan/arabinoxylan or to identify and deselect plants with decreased fructan/arabinoxylan. Wheat plants generated by the methods of the invention are also a feature of the invention.

### CROSS REFERENCE TO RELATED APPLICATIONS

This Application claims priority from, and benefit of, U.S. Provisional Application 62/221,348 filed on Sep. 21, 2015 and U.S. Provisional Application 62/377,763 filed on Aug. 22, 2016. The entire contents of these applications is hereby incorporated by reference into this Application.

### REFERENCE TO SEQUENCE LISTING SUBMITTED ELECTRONICAL

The official copy of the sequence listing is submitted electronically via EFS-Web as an ASCII formatted sequence listing with a file named “77888_ST25”, created on Sep. 12, 2016, and having a size of 3.92 kilobytes, and is filed concurrently with the specification. The sequence listing contained in this ASCII formatted document is part of the specification, and is incorporated herein by reference in its entirety.

### FIELD OF THE INVENTION

The present invention relates to methods useful in increasing major components of dietary fiber, fructan and/or arabinoxylan, in wheat plants.

### BACKGROUND OF THE INVENTION

The benefits of high dietary fiber in food have been appreciated since at least the 5^{th} century BC when Hippocrates described that coarse wheat had improved laxative effect relative to refined wheat. J. H. Kellogg advocated the use of bran to improve digestive function and prevent disease in the 1920s. Fifty years later, Denis Burkitt revived scientific and popular interest in the beneficial properties of dietary fiber as protective against traditional Western diseases such as diabetes, cardiovascular disease, and obesity. Even more recent studies have found a robust correlation between consumption of high fiber foods and reduced risk of cardiovascular disease. See e.g., Slavin, J., 2013, Nutrients 5(4): 1417-35.

Packaged food labels in many countries disclose the amount of dietary fiber present in the food. The European Union and its member countries regulate whether a food can be labeled as a “source” of fiber or is “high fibre.” Similarly, in the United States, the Food and Drug Administration (FDA) regulates and imposes specific requirements for grain products labeled as being “high” in fiber or a “good source of” fiber. In certain cases, the FDA also allows certain grain products that meet specified requirements for fiber and other nutritional content to indicate that such grain products may reduce some types of cancer.

Dietary fiber includes two main types: insoluble and soluble. Insoluble fiber does not dissolve in water. Some types of insoluble fiber are not fermented by intestinal bacteria and help promote bowel activity. Other types of insoluble fiber, such as resistant starch, can be fully fermented by large intestinal bacteria and are associated with reduced risk of diabetes, lower glycemic index, and increased insulin sensitivity.

Soluble dietary fiber dissolves in water and can be fermented by intestinal bacteria, leading to the production of healthful compounds including short chain fatty acids. Soluble dietary fiber includes fructans; oligosaccharide polymers that contain fructose. Fructans have been shown to increase beneficial bifidobacteria, which are associated with reduced colonic disorders such as constipation, hemorrhoids, and colonic cancer. See, e.g., Slavin, J., 2013, at 1425-26. Other soluble fibers (e.g., beta-glucan, psyllium, pectin, and guar gum) in foods (such as oats, barley, and psyllium) have been shown to decrease serum levels of low density lipoprotein (LDL) without affecting high density lipoprotein (HDL). See Slavin, J., 2013, at 1428. Furthermore, enhancement of prebiotic soluble fiber of the short- and long-chain insulin-type fructans has been found to enhance calcium absorption and bone mineralization in both animals and human adolescents. See Abrams et al., 2005, Am J. Clin. Nutrition 82(2): 471-76.

High levels of fructan are found in certain commercial crops such as chicory (42%), Jerusalem artichoke (18%), dandelion greens (14%), and garlic (13%). Leeks, onions, globe artichoke and onion have from 4% to 7% fructan content. Staple grain crops tend to have much lower fructan content, which typically ranges from 0.7% in rye and 0.8% in barley to 2.5% in whole meal wheat. Although white flour normally has 0.2% to 1.8% fructan, in the American diet, the largest source of fructan intake is from wheat (69%), followed by onion (23%), bananas (3%), garlic (2%), and other sources (2%). Moshfegh et al, 1999, J. Nutrition 129(7): 1407s-1411s. Thus, despite its relatively low fructan content, wheat represents the largest source of fructan for Americans.

Several genes have been identified that are involved in fructan biosynthesis. These include 1-SST, 1-FFT, and 6-SFT. Genetic markers and quantitative trait loci (QTL) have been found to be associated with different components of dietary fiber. One study has identified a major QTL affecting fructan accumulation in a set of diverse wheat lines. See Bao-Lam et al., 2012, Plant Mol. Biol. 80: 299-314. In other studies, analysis of mapping populations identified QTLs associated with arabinoxylan content in wheat. Lafiandra et al. 2014, J. Cell Science 59: 312-326, at 321. Still other studies evaluated the interaction of environmental and genetic factors and have found that, although highly heritable components affected by different environments were observed on different sites in different years, (i) such highly heritable factors are viable targets for plant breeders to develop novel plant varieties with enhanced health benefits and (ii) varieties with increased dietary fiber are preferably combined with good agronomic performance yield and/or qualities suitable for grain end-products (e.g., milling). See Shewry et al. 2010, J Agric Food Chem. 58: 9291-98, at 9297.

Arabinoxylan from cereals, including wheat, is a major source of dietary fiber for humans. Several studies have found correlations between arabinoxylan intake and the following health benefits: blood sugar control in diabetics and non-diabetics and improved gut-health and diminished constipation, and lower cholesterol. In one study, patients with hepatocellular carcinoma that were treated with a combination of arabinoxylan and interventional chemotherapy had lower incidence of relapse, longer survival, and greater decrease in tumor volumes than patients treated with chemotherapy alone. Bang et al, 2010, Anticancer Res. 30(12): 5145-51.

There is a desire to create diets and food products that deliver higher levels of fructan and/or arabinoxylan. There is also a desire for novel markers, QTLs, and breeding methods to develop grain and grain products that deliver more fructan and/or arabinoxylan.

### SUMMARY OF THE INVENTION

The disclosed invention is based, at least in part, on the discovery of markers for increased fructan and/or arabinoxlyan. The invention discloses that these markers can be used to create new wheat varieties that have a combination of (i) two or more disclosed markers and have increased levels fructan and/or arabinoxlyan, (ii) three or more disclosed markers and have further increased levels fructan and/or arabinoxlyan, (iii) four or more disclosed markers and have even further increased fructan and/or arabinoxlyan relative to comparable wheat variety having only three disclosed markers, and (iv) five or more disclosed markers and have additionally increased fructan and/or arabinoxlyan relative to comparable wheat variety having only four disclosed markers. Accordingly, the invention provides methods for planting crops of wheat having increased fructan/arabinoxlyan, which are useful, e.g., to make wheat flour having higher levels of fructan and/or arabinoxylan

In one aspect, the invention provides a method of identifying a wheat plant that displays increased fructan/arabinoxlyan (hereinafter fructan/arabinoxylan), comprising detecting in wheat tissue one or more alleles of quantitative marker loci disclosed herein. The markers and alleles associated with increased fructan/arabinoxylan are located within chromosomal intervals flanked by the left and right interval markers disclosed herein. Thus, the invention provides (i) QTL 1A which comprises and is flanked by left interval markers and right interval markers on chromosome 1A, (ii) QTL 1B which comprises and is flanked by left interval markers and right interval markers on chromosome 1B, (iii) QTL 2B-1 which comprises and is flanked left interval markers and right interval markers on chromosome 2B, (iv) QTL 2B-2 which comprises and is flanked left interval markers and right interval markers on chromosome 2B, (v) QTL 2D which comprises and is flanked by left interval markers and right interval markers on chromosome 2D, (vi) QTL 6B which comprises and is flanked by left interval markers and right interval markers on chromosome 6B, (vii) QTL 7A-1 which comprises and is flanked by left interval markers and right interval markers on chromosome 7A, (viii) QTL 7A-2 which comprises and is flanked by left interval markers and right interval markers on chromosome 7A, and (ix) QTL 7B which comprises and is flanked by left interval markers and right interval markers on chromosome 7B. Therefore, given a population of wheat plants having an average content fructan/arabinoxylan, the invention provides a method for selecting from within that population plants having a higher content of fructan/arabinoxylan. Such selected plants can be used in a breeding program to create progeny plants or new varieties of wheat plants that have a higher content of fructan/arabinoxylan. Such breeding methods are discussed in more detail herein. Examples of left and right interval markers for each of the foregoing QTLs are provided in Table 1; each marker is identified as single nucleotide polymorphism (SNP), the trait that each QTL is diagnostic for is indicated by “sAX” for increased soluble arabinoxylan, “tAX” for increased total arabinoxylan, and “Fruc” for increased fructan, and the relative chromosomal location for each interval marker is provided as a linkage distance “dist” in centimorgans (cM).

TABLE 1

| QTL | | Left Interval | dist | Right Interval |
|---|---|---|---|---|
| (Chr) | Trait | Marker | (cM) | Marker |
| 1A | sAX, | Kukri_c54467_100 | 110.42-122.39 | BS00077504_51 |
| | tAX | | | |
| 1B | sAX, | RAC875_c24429_132 | 113.58-115.97 | wsnp_BE443531B_Ta_1_1 |
| | tAX | | | |
| 2B-1 | tAX | BobWhite_c6472_601 | 83.91-89.56 | Kukri_c11864_500 |
| 2B-2 | Fruc | Excalibur_c6502_397 | 158.11-160.35 | RAC875_c34516_316 |
| 2D | Fruc | TA012840-0369 | 51.67-57.12 | wsnp_Ex_c25311_34578436 |
| 6B | sAX, | wsnp_Ex_c26172_35422935 | 81.12-91.9 | darts_wPt-8892 |
| | tAX | | | |
| 7A-1 | Fruc | Excalibur_c30730_253 | 42.69-43.28 | BS00065529_51 |
| 7A-2 | sAX | Ex_c9556_2547 | 82.53-89.21 | Excalibur_c4759_564 |
| 7B | tAX | Ex_c101666_634 | 153.22-177.53 | Excalibur_c3423_1170 |

In certain embodiments, the method further includes detecting one or more of the foregoing marker loci in combination with a QTL 7A-3 which comprises the locus on chromosome 7A known to include 1-SST, 1-FFT, and 6-SFT genes described in Bao-Lam et al., 2012. Examples of left and right interval markers for each of the foregoing QTL 7A-3 are provided in Table 2.

TABLE 2

| QTL | | Left Interval | dist | Right Interval |
|---|---|---|---|---|
| (Chr) | Trait | Marker | (cM) | Marker |
| 7A-3 | Fruc | Excalibur_c24750_504 | 175.11-181.46 | darts_wPt- |
| | | | | 0808 |

Thus, the invention provides a method of identifying a wheat plant that displays increased fructan/arabinoxylan, comprising detecting in wheat tissue a marker allele (e.g., a SNP) associated with increased fructan/arabinoxylan, which marker is located within one or more of the left and right intervals disclosed in Table 1 for QTL 1A, QTL 1B, QTL 2B-1, QTL 2B-2, QTL 2D, QTL 6B, QTL 7A-1, QTL 7A-2, and QTL 7B. In certain embodiments, the method further includes detecting one or more alleles (e.g., SNPs) associated with increased fructan/arabinoxylan which are located within the left and right intervals disclosed in Table 2 for QTL 7A-3 in combination with one or more of the foregoing marker alleles located QTL 1A, QTL 1B, QTL 2B-1, QTL 2B-2, QTL 2D, QTL 6B, QTL 7A-1, QTL 7A2, and QTL 7B that are also associated with increased fructan/arabinoxylan.

In particular examples, the methods of identifying or selecting a wheat plant that displays increased fructan/arabinoxylan disclosed herein can include detecting in wheat tissue one or more SNP markers located within one or more of the left and right intervals disclosed in Table 3. The SNP markers for QTL 2D and QTL 7B represent a haplotype of two or three SNP alleles, respectively, and when these SNPs markers are detected together they are indicative of increased fructan/arabinoxlyan.

TABLE 3

| | | | dist |
|---|---|---|---|
| QTL | Trait | Marker | (cM) |
| 1A | sAX, | wsnp_Ex_c3572_6531810, | 110.42-115.15 |
| | tAX | wsnp_Ex_c3142_5808713 | |
| 1B | sAX, | wsnp_Ex_c10869_17671164, | 114.21 |
| | tAX | IAAV1364, | |
| | | BobWhite_c4147_1351 | |
| 2B-1 | tAX | BobWhite_c6472_601, | 83.91-84.01 |
| | | RAC875_c96504_413 | |
| 2B-2 | Fruc | Excalibur_c6502_397, | 158.11-160.06 |
| | | Excalibur_c25234_143 | |
| 2D | Fruc | Haplotype: | 55.67-55.08 |
| | | (i) TA012840-0369 | |
| | | and (ii) one of (BS00000905_51, | |
| | | wsnp_Ex_c25311_34578436) | |
| 6B | sAX, | wsnp_Ku_c11422_18657479, | 84.1-87.99 |
| | tAX | Ku_c2212_411 | |
| 7A-1 | Fruc₁ | Excalibur_c30730_253, | 42.69-43.28 |
| | | BS00065529_51 | |
| 7A-2 | sAX | RAC875_c32212_84, | 85.25-85.33 |
| | | IACX3013 | |
| 7A-3 | Fruc₂ | BS00085135_51, | 175.19-175.25 |
| | | Tdurum_contig52015_ 1090 | |
| 7B | tAX | Haplotype: | 153.2-164.54 |
| | | (i) one of (Ex_c101666_634, | |
| | | wsnp_Ex_c2103_3947695) and | |
| | | (ii) one of (GENE-4833_102, | |
| | | snp90kbi_Kukri_c67849_109) and | |
| | | (iii) one of (Excalibur_c40122_280, | |
| | | snp90kbi_BobWhite_c44404_312, | |
| | | snp90kpoly_Tdurum_contig31682_53) | |

In some embodiments, the invention provides a method for identifying wheat plants with increased fructan/arabinoxylan that includes detecting a panel of QTLs which is diagnostic for one type of fiber. For example, the method can include identifying a wheat plant that includes screening for a panel of markers that includes one or more markers located within each of QTLs 2B-2, 2D, 7A-1, and 7A-3 disclosed which are indicative of increased fructan (“Fruc”). In another example, the method can include identifying a wheat plant that includes screening for a panel of markers that includes one or more markers located within each of QTLs 1A, 1B, 6B, and 7A-2, which are indicative of soluble arabinoxylan (“sAX”). In yet another example, the method can include identifying a wheat plant that includes screening for a panel of markers that includes one or more markers located within each of QTLs 1A, 1B, 2B-1, 6B, 7A-2, and 7B which are indicative of total arabinoxylan (“tAX”). In still another example, the panel can include a combination of at least one, two, three, or four markers from each of the foregoing disclosed panels for fructan, soluble arabinoxylan, and total arabinoxylan. The method can include screening for a panel of markers that includes all of the markers located within each disclosed panel for fructan, soluble arabinoxylan, and total arabinoxylan.

In another aspect, the invention provides a method of introgressing one or more disclosed QTL for increased fructan/arabinoxlyan into progeny plants. The method includes (i) selecting at least one wheat plant having one or more markers for increased fructan/arabinoxlyan, wherein each marker is located within QTL 1A, QTL 1B, QTL 2B-1, QTL 2B-2, QTL 2D, QTL 6B, QTL 7A-1, QTL 7A-2, or QTL 7B, (ii) crossing the selected wheat plant(s) with at least one second wheat plant to create progeny plants, (iii) evaluating progeny plants for one or more of the markers for increased fructan/arabinoxlyan located on one or more of QTL 1A, QTL 1B, QTL 2B-1, QTL 2B-2, QTL 2D, QTL 6B, QTL 7A-1, QTL 7A-2, and QTL 7B, and (iv) selecting progeny plants into which the one or more of the markers for increased fructan/arabinoxlyan are introgressed. In certain embodiments, the method includes (i) selecting at least one wheat plant having at least one marker in QTL 7A-3 for increased fructan/arabinoxlyan in combination with at least one marker for increased fructan/arabinoxlyan located, each marker located within QTL 1A, QTL 1B, QTL 2B-1, QTL 2B-2, QTL 2D, QTL 6B, QTL 7A-1, QTL 7A-2, or QTL 7B, (ii) crossing the selected wheat plant(s) with at least one second wheat plant to create progeny plants, (iii) evaluating progeny plants for one or more of the markers for increased fructan/arabinoxlyan located on QTL 7A-3 and on one or more of QTL 1A, QTL 1B, QTL 2B-1, QTL 2B-2, QTL 2D, QTL 6B, QTL 7A-1, QTL 7A-2, and QTL 7B, and (iv) selecting progeny plants into which the evaluated markers for increased fructan/arabinoxlyan are introgressed. In further embodiments of the foregoing methods of introgressing one or more disclosed QTL for increased fructan/arabinoxlyan, the second parental wheat plant includes one or more desirable traits selected from the group consisting of high grain yield, good end-use quality (e.g., good milling, good flour, or good baking qualities), disease resistance, pest resistance, herbicide tolerance, and tolerance to abiotic stresses (e.g., mineral, moisture, drought and heat tolerance); and the selected progeny plant includes the one or more disclosed QTL for increased fructan/arabinoxlyan as well as the one or more desirable traits from the parental second wheat plant. For example, the second wheat plant can be an elite commercial variety and progeny plants can be selected that possess the one or more alleles from the first wheat plant and also have desirable agronomic traits and/or end-use qualities from the second plant.

The invention provides a wheat progeny plant produced by any method of introgressing one or more QTLs for increased fructan/arabinoxlyan disclosed herein.

In yet another aspect, the invention provides a selfing method that includes selecting a wheat plant having one or more markers for increased fructan/arabinoxlyan, wherein the one or more markers are located on one or more of QTL 1A, QTL 1B, QTL 2B-1, QTL 2B-2, QTL 2D, QTL 6B, QTL 7A-1, QTL 7A-2, and QTL 7B and then selfing (inbreeding) the selected wheat plant to produce a progeny population comprising the one or more markers for increased fructan/arabinoxlyan. In a further embodiment, the method includes selecting a wheat plant having at least one marker in QTL 7A-3 for increased fructan/arabinoxlyan in combination with at least one marker for increased fructan/arabinoxlyan located in one or more of QTL 1A, QTL 1B, QTL 2B-1, QTL 2B-2, QTL 2D, QTL 6B, QTL 7A-1, QTL 7A-2, and QTL 7B and then selfing (inbreeding) the selected wheat plant to produce a progeny population comprising the one or more markers in QTL 7A-3 and the one or more of QTL 1A, QTL 1B, QTL 2B-1, QTL 2B-2, QTL 2D, QTL 6B, QTL 7A-1, QTL 7A-2, and QTL 7B markers for increased fructan/arabinoxlyan.

The invention provides a wheat progeny plant produced by any method of selfing a wheat plant with one or more QTLs for increased fructan/arabinoxlyan disclosed herein.

In still another aspect, the invention provides a wheat plant or a wheat crop comprising plants having one or more of the QTL 1A, QTL 1B, QTL 2B-1, QTL 2B-2, QTL 2D, QTL 6B, QTL 7A-1, QTL 7A-2, and QTL 7B markers for increased fructan/arabinoxlyan discloses herein, which are flanked by left interval markers and right interval markers disclosed in Table 1. In some embodiments, the wheat plant or wheat crop of the invention comprises two, three, four, five, six, seven, eight, nine, or ten distinct markers and each distinct marker is located within QTL 1A, QTL 1B, QTL 2B-1, QTL 2B-2, QTL 2D, QTL 6B, QTL 7A-1, QTL 7A-2, or QTL 7B. For example, the wheat plant of the invention comprises at least one marker in QTL 7A-3 for increased fructan/arabinoxlyan in combination with two markers, three markers, four markers, five markers, six markers, seven markers, eight markers, nine markers, or ten markers for increased fructan/arabinoxlyan located in one or more of QTL 1A, QTL 1B, QTL 2B-1, QTL 2B-2, QTL 2D, QTL 6B, QTL 7A-1, QTL 7A-2, and QTL 7B. In particular examples, the wheat plant of the invention comprises one or more markers located within each of QTLs 2B-2, 2D, 7A-1, and 7A-3 disclosed which are indicative of increased fructan, one or more markers located within each of QTLs 1A, 1B, 6B, and 7A-2, which are indicative of soluble arabinoxylan (“sAX”), or one or more markers located within each of QTLs 1A, 1B, 2B-1, 6B, 7A-2, and 7B which are indicative of total arabinoxylan (“tAX”).

In certain embodiments, the invention provides a method of generating a wheat crop. The method includes planting a field with wheat seed that has one or more alleles of a marker locus for increased fructan/arabinoxylan, wherein each marker locus is located is located within a chromosomal interval flanked by a left and right interval marker for QTL 1A, QTL 1B, QTL 2B-1, QTL 2B-2, QTL 2D, QTL 6B, QTL 7A-1, QTL 7A-2, or QTL 7B; growing wheat plants from the planted wheat seed; and optionally harvesting the wheat plants from the field, thereby generating a wheat crop. In some embodiments, the wheat seed planted to generate the wheat crop of the invention comprises two, three, four, five, six, seven, eight, nine, or ten distinct alleles, each located in a marker locus within QTL 1A, QTL 1B, QTL 2B-1, QTL 2B-2, QTL 2D, QTL 6B, QTL 7A-1, QTL 7A-2, or QTL 7B.

The invention also provides wheat seed units (e.g. seed bags, packages, or lots), which can be planted and used in the method of generating a crop disclosed herein. At least 50%, at least 60%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% of the seed contained by such a unit (e.g., in a seed bag, package, or lot) has two, three, four, five, six, seven, eight, nine, or ten distinct alleles for increased fructan/arabinoxlyan, each allele located in marker locus within QTL 1A, QTL 1B, QTL 2B-1, QTL 2B-2, QTL 2D, QTL 6B, QTL 7A-1, QTL 7A-2, or QTL 7B. In certain examples, the wheat seed contained by the unit (and which can be planted to generate a crop) comprise at least one marker in QTL 7A-3 for increased fructan/arabinoxlyan in combination with two markers, three markers, four markers, five markers, six markers, seven markers, eight markers, nine markers, or ten distinct alleles for increased fructan/arabinoxlyan, each located in QTL 1A, QTL 1B, QTL 2B-1, QTL 2B-2, QTL 2D, QTL 6B, QTL 7A-1, QTL 7A-2, or QTL 7B. In particular examples, the units contain wheat seed having one or more markers located within each of QTLs 2B-2, 2D, 7A-1, and 7A-3 and can produce a crop having increased fructan, units containing wheat seed having one or more markers located within each of QTLs 1A, 1B, 6B, and 7A-2 can produce a crop having increased soluble arabinoxylan (“sAX”), and units containing wheat seed having or one or more markers located within each of QTLs 1A, 1B, 2B-1, 6B, 7A-2, and 7B can produce a crop having increased total arabinoxylan (“tAX”).

In particular embodiments, the first wheat plant is crossed with a second wheat plant that has desirable agronomic traits Such wheat plants selected by this method are also of interest.

### BRIEF DESCRIPTION OF FIGURES AND SEQUENCE LISTINGS

The invention can be more fully understood from the following detailed description and the accompanying drawings and Sequence Listing which form a part of this application.

FIG. 1 is a set of three histograms showing results of analyzing total arabinoxylan variation in wholemeal or white flour prepared from four-way multiparent advanced generation inter-cross (“MAGIC”) wheat populations grown at different locations.

FIG. 2 is a set of three histograms showing results of analyzing soluble arabinoxylan variation in wholemeal or white flour prepared from four-way MAGIC populations grown at different locations.

FIG. 3 is a pair of histograms showing results of analyzing fructan variation in wholemeal prepared from four-way multiparent advanced generation inter-cross (“MAGIC”) populations grown at different locations.

FIG. 4 is a pair of graphs showing the genomic estimated breeding value (“GEBV”) of lines having the indicated number (0-4) of different QTL markers disclosed herein for increased fructan.

FIG. 5 is a set of graphs showing the GEBV for of lines having the indicated number (0-5) of different QTL markers disclosed herein for increased soluble arabinoxylan (sAX) or increased total arabinoxylan (tAX).

atcccttgcgacaaaagcXaa (SEQ ID NO:1), wherein Xaa is T or G, is a forward primer for amplification of a QTL 1A marker (90K chip index: 77717)(SNP ID: IWA3339).

gggcatttaagacatggtatggXaa (SEQ ID NO:2), wherein Xaa is T or G, is a forward primer for amplification of a QTL 1A marker (90K chip index: 77870)(SNP ID: IWA3536).

tggaattcctcctgctccXaa (SEQ ID NO:3), wherein Xaa is A or G, is a forward primer for amplification of a QTL 1B marker (90K chip index: 3175)(SNP ID: IWB3175).

tgtcctgcttcttcccagtXaa (SEQ ID NO:4), wherein Xaa is T or C, is a forward primer for amplification of a QTL 1B marker (90K chip index: 3176)(SNP ID: IWB3176).

ctacattggccatcacacaggaXaa (SEQ ID NO:5), wherein Xaa is T or C, is a forward primer for amplification of a QTL 1B marker (90K chip index: 52095)(SNP ID: IWB52095).

cggtcattctttcagaaagcatctXaa (SEQ ID NO:6), wherein Xaa is T or C, is a forward primer for amplification of a QTL 1B marker (90K chip index: 52095)(SNP ID: IWB52095).

gagtttgacttgatcccgagXaa (SEQ ID NO:7), wherein Xaa is A or G, is a forward primer for amplification of a QTL 2B-2 marker (90K chip index: 24280)(SNP ID: IWB24280).

cacgcttcatgtttttctccXaa (SEQ ID NO:8), wherein Xaa is A or G, is a forward primer for amplification of a QTL 2B-2 marker (90K chip index: 28342)(SNP ID: IWB28342).

cacgcttcatgtttttctccXaa (SEQ ID NO:9), wherein Xaa is T or C, is a forward primer for amplification of a QTL 2D marker (90K chip index: 27678)(SNP ID: IWB27678).

gtcacatcgtttattaaccgcXaa (SEQ ID NO:10), wherein Xaa is A or G, is a reverse primer for amplification of a QTL 2D marker (90K chip index: 77420)(SNP ID: IWA2961).

tgtcgcacAcctagttgtctgtaaXaa (SEQ ID NO:11), wherein Xaa is T or C, is a reverse primer for amplification of a QTL 6B marker (90K chip index: 38811)(SNP ID: IWB38811).

agcagtctccacgtagcXaa (SEQ ID NO:12), wherein Xaa is T or C, is a reverse primer for amplification of a QTL 6B marker (90K chip index: 80025)(SNP ID: IWA6420).

gaagatcccaccacttgacXaa (SEQ ID NO:13), wherein Xaa is T or C, is a reverse primer for amplification of a QTL 7A-1 marker (90K chip index: 44281)(SNP ID: IWA44281).

tgaacggaagctgctccXaa (SEQ ID NO:14), wherein Xaa is T or C, is a reverse primer for amplification of a QTL 7A-1 marker (90K chip index: 21840)(SNP ID: IWA21840).

acaatcaccgctggcttcXaa (SEQ ID NO:15), wherein Xaa is T or C, is a reverse primer for amplification of a QTL 7A-2 marker (90K chip index: 56709)(SNP ID: IWA56709).

aatggtttttgtgtgagttctgXaa (SEQ ID NO:16), wherein Xaa is A or G, is a reverse primer for amplification of a QTL 7A-2 marker (90K chip index: 8231)(SNP ID: IWA8231).

gcaccgtcagcaaggacXaa (SEQ ID NO:17), wherein Xaa is T or C, is a reverse primer for amplification of a QTL 7A-3 marker (90K chip index: 11397)(SNP ID: IWB 11397).

gcaccgtcagcaaggacXaa (SEQ ID NO:18), wherein Xaa is A or G, is a reverse primer for amplification of a QTL 7A-3 marker (90K chip index: 72227)(SNP ID: IWB72227).

gtttgtttgatcctGttaaggctaXaa (SEQ ID NO:19), wherein Xaa is T or G, is a reverse primer for amplification of a QTL 7B marker (90K chip index: 19554)(SNP ID: IWB 19554.

cacctctaggatggaaatagcaaXaa (SEQ ID NO:20), wherein Xaa is A or G, is a reverse primer for amplification of a QTL 7B marker (90K chip index: 34191)(SNP ID: IWB34191).

### DETAILED DESCRIPTION OF THE INVENTION

The present invention provides methods for identifying and selecting wheat plants with increased fructan/arabinoxylan. The following definitions are provided as an aid to understand the invention.

The term “additive effect” is calculated by the following equation: Additive effect=“Elite line effect”−“Donor line effect”.

A negative additive effect indicates that the QTL comes from the donor; alternatively, a positive additive effect indicates that it comes from the elite line.

The term “allele” refers to one of two or more different nucleotide sequences that occur at a specific locus.

An “amplicon” is an amplified nucleic acid, e.g., a nucleic acid that is produced by amplifying a template nucleic acid by any available amplification method (e.g., PCR, LCR, transcription, or the like).

The term “amplifying” in the context of nucleic acid amplification is any process whereby additional copies of a selected nucleic acid for a transcribed form thereof are produced. Typical amplification methods include various polymerase based replication methods, including the polymerase chain reaction (PCR), ligase mediated methods such as the ligase chain reaction (LCR) and RNA polymerase based amplification (e.g., by transcription) methods.

The term “assemble” applies to BACs and their propensities for coming together to form contiguous stretches of DNA. A BAC “assembles” to a contig based on sequence alignment, if the BAC is sequenced, or via the alignment of its BAC fingerprint to the fingerprints of other BACs. The assemblies can be found using publicly available databases and tools on the internet.

An allele is “associated with” a trait when it is linked to it and when the presence of the allele is an indicator that the desired trait or trait form will occur in a plant comprising the allele.

A “BAC”, or bacterial artificial chromosome, is a cloning vector derived from the naturally occurring F factor of Escherichia coli. BACs can accept large inserts of DNA sequence. In wheat, a number of BACs, or bacterial artificial chromosomes, each containing a large insert of wheat genomic DNA, have been assembled into contigs (overlapping contiguous genetic fragments, or “contiguous DNA”).

“Backcrossing” refers to the process whereby progeny are repeatedly crossed back to one of the parents. In a backcrossing scheme, the “donor” parent refers to the parental plant with the desired gene or locus to be introgressed. The “recipient” parent (used one or more times) or “recurrent” parent (used two or more times) refers to the parental plant into which the gene or locus is being introgressed. For example, see Ragot, M. et al. (1995) Marker-assisted backcrossing: a practical example, in Techniques et Utilisations des Marqueurs Moleculaires Les Colloques, Vol. 72, pp. 45-56, and Openshaw et al., (1994) Marker-assisted Selection in Backcross Breeding, Analysis of Molecular Marker Data, pp. 41-43. The initial cross gives rise to the F1 generation: the term “BC1” then refers to the second use of the recurrent parent, “BC2” refers to the third use of the recurrent parent, and so on.

A centimorgan (“cM”) is a unit of measure of recombination frequency. One cM is equal to a 1% chance that a marker at one genetic locus will be separated from a marker at a second locus due to crossing over in a single generation.

The “90K chip” is the genotyping array that includes about 90,000 gene-associated SNPs described in Wang et al., 2014, Plant Biotech J. 12: 787-796.

“Chromosomal interval” designates a contiguous linear span of genomic DNA that resides in planta on a single chromosome. The genetic elements or genes located on a single chromosomal interval are physically linked. The size of a chromosomal interval is not particularly limited. In some aspects, the genetic elements located within a single chromosomal interval are genetically linked, typically with a genetic recombination distance of, for example, less than or equal to 20 cM, or alternatively, less than or equal to 10 cM. That is, two genetic elements within a single chromosomal interval undergo recombination at a frequency of less than or equal to 20% or 10%.

The term “chromosomal interval” designates any and all intervals defined by any of the markers set forth in this invention. Chromosomal intervals that correlate with increased fructan/arabinoxylan are provided. These intervals, are located on the chromosomes and flanked by interval markers identified in Table 1, herein.

The term “complement” refers to a nucleotide sequence that is complementary to a given nucleotide sequence, i.e., the sequences are related by the base-pairing rules.

The term “contiguous DNA” refers to overlapping contiguous genetic fragments.

The term “crop” means an intentionally cultivated plurality of plants, e.g., wheat plants for use in commerce, feed, or food. A crop refers to such plants while in their growing location (e.g., field or greenhouse) and also after the plants are gathered, harvested, and optionally treated or processed prior to their end use.

The term “crossed” or “cross” means the fusion of gametes via pollination to produce progeny (e.g., cells, seeds or plants). The term encompasses both sexual crosses (the pollination of one plant by another) and selfing (self-pollination, e.g., when the pollen and ovule are from the same plant). The term “crossing” refers to the act of fusing gametes via pollination to produce progeny.

A “favorable allele” is the allele at a particular locus that confers, or contributes to, a desirable phenotype, e.g., increased fructan/arabinoxylan, or alternatively, is an allele that allows the identification of plants with decreased fructan that can be removed from a breeding program or planting (“counterselection”). A favorable allele of a marker is a marker allele that segregates with the favorable phenotype, or alternatively, segregates with the unfavorable plant phenotype, therefore providing the benefit of identifying plants.

“Fragment” is intended to mean a portion of a nucleotide sequence. Fragments can be used as hybridization probes or PCR primers using methods disclosed herein.

A “genetic map” is a description of genetic linkage relationships among loci on one or more chromosomes within a given species, generally depicted in a diagrammatic or tabular form. For each genetic map, distances between loci are measured by the recombination frequencies between them, and recombinations between loci can be detected using a variety of molecular genetic markers (also called molecular markers). A genetic map is a product of the mapping population, types of markers used, and the polymorphic potential of each marker between different populations. The order and genetic distances between loci can differ from one genetic map to another. However, information such as marker position and order can be correlated between maps by determining the physical location of the markers on the chromosome of interest, using the B73 reference genome, version 2, which is publicly available on the internet. One of ordinary skill in the art can use the publicly available genome browser to determine the physical location of markers on a chromosome.

The term “genetic marker” shall refer to any type of nucleic acid based marker, including but not limited to, Restriction Fragment Length Polymorphism (RFLP), Simple Sequence Repeat (SSR), Random Amplified Polymorphic DNA (RAPD), Cleaved Amplified Polymorphic Sequences (CAPS) (Rafalski and Tingey, 1993, Trends in Genetics 9:275-280), Amplified Fragment Length Polymorphism (AFLP) (Vos et al, 1995, Nucleic Acids Res. 23:4407-4414), Single Nucleotide Polymorphism (SNP) (Brookes, 1999, Gene 234:177-186), Sequence Characterized Amplified Region (SCAR) (Pecan and Michelmore, 1993, Theor. Appl. Genetics., 85:985-993), Sequence Tagged Site (STS) (Onozaki et al. 2004, Euphytica 138:255-262), Single Stranded Conformation Polymorphism (SSCP) (Orita et al., 1989, Proc. Nat'l. Acad. Sci. USA 86:2766-2770), Inter-Simple Sequence Repeat (ISR) (Blair et al. 1999, Theor. Appl. Genetics 98:780-792), Inter-Retrotransposon Amplified Polymorphism (IRAP), Retrotransposon-Microsatellite Amplified Polymorphism (REMAP) (Kalendar et al., 1999, Theor. Appl. Genetics 98:704-711), an RNA cleavage product (such as a Lynx tag), and the like.

“Genetic recombination frequency” is the frequency of a crossing over event (recombination) between two genetic loci. Recombination frequency can be observed by following the segregation of markers and/or traits following meiosis.

“Genome” refers to the total DNA, or the entire set of genes, carried by a chromosome or chromosome set.

The term “genotype” is the genetic constitution of an individual (or group of individuals) at one or more genetic loci, as contrasted with the observable trait (the phenotype). Genotype is defined by the allele(s) of one or more known loci that the individual has inherited from its parents. The term genotype can be used to refer to an individual's genetic constitution at a single locus, at multiple loci, or, more generally, the term genotype can be used to refer to an individual's genetic make-up for all the genes in its genome.

“Germplasm” refers to genetic material of or from an individual (e.g., a plant), a group of individuals (e.g., a plant line, variety or family), or a clone derived from a line, variety, species, or culture. The germplasm can be part of an organism or cell, or can be separate from the organism or cell. In general, germplasm provides genetic material with a specific molecular makeup that provides a physical foundation for some or all of the hereditary qualities of an organism or cell culture. As used herein, germplasm includes cells, seed or tissues from which new plants may be grown, or plant parts, such as leafs, stems, pollen, or cells that can be cultured into a whole plant.

A “haplotype” is the genotype of an individual at a plurality of genetic loci, i.e. a combination of alleles. Typically, the genetic loci described by a haplotype are physically and genetically linked, i.e., on the same chromosome segment. The term “haplotype” can refer to sequence, polymorphisms at a particular locus, such as a single marker locus, or sequence polymorphisms at multiple loci along a chromosomal segment in a given genome. The former can also be referred to as “marker haplotypes” or “marker alleles”, while the latter can be referred to as “long-range haplotypes”.

The “heritability (h2)” of a trait within a population is the proportion of observable differences in a trait between individuals within a population that is due to genetic differences. The h2 value of the QTL is a percentage of variation that is explained by genetics, instead of environment.

The term “heterozygous” means a genetic condition wherein different alleles reside at corresponding loci on homologous chromosomes.

The term “homozygous” means a genetic condition wherein identical alleles reside at corresponding loci on homologous chromosomes.

“Hybridization” or “nucleic acid hybridization” refers to the pairing of complementary RNA and DNA strands as well as the pairing of complementary DNA single strands.

The term “hybridize” means the formation of base pairs between complementary regions of nucleic acid strands.

The term “indel” refers to an insertion or deletion, wherein one line may be referred to as having an insertion relative to a second line, or the second line may be referred to as having a deletion relative to the first line.

The term “introgression” or “introgressing” refers to the transmission of a desired allele of a genetic locus from one genetic background to another. For example, introgression of a desired allele at a specified locus can be transmitted to at least one progeny via a sexual cross between two parents of the same species, where at least one of the parents has the desired allele in its genome. Alternatively, for example, transmission of an allele can occur by recombination between two donor genomes, e.g., in a fused protoplast, where at least one of the donor protoplasts has the desired allele in its genome. The desired allele can be, e.g., a selected allele of a marker, a QTL, a transgene, or the like. In any case, offspring comprising the desired allele can be repeatedly backcrossed to a line having a desired genetic background and selected for the desired allele, to result in the allele becoming fixed in a selected genetic background. For example, one or more favorable alleles of QTL 2A, QTL 2B, QTL 2D, QTL 4A, and QTL 7D described herein may be introgressed into a recurrent parent that (prior to introgression) displays low or normal fructose content. The recurrent parent line with the introgressed gene or locus then has improved fructan content.

As used herein, the term “linkage” is used to describe the degree with which one marker locus is associated with another marker locus or some other locus (for example, a QTL 2A, QTL 2B, QTL 2D, QTL 4A, or QTL 7D locus). The linkage relationship between a molecular marker and a phenotype is given as a “probability” or “adjusted probability”. Linkage can be expressed as a desired limit or range. For example, in some embodiments, any marker is linked (genetically and physically) to any other marker when the markers are separated by less than 50, 40, 30, 25, 20, or 15 map units for cM). In some aspects, it is advantageous to define a bracketed range of linkage, for example, between 10 and 20 cM, between 10 and 30 cM, or between 10 and 40 cM. The more closely a marker is linked to a second locus, the better an indicator for the second locus that marker becomes. Thus, “closely linked loci” such as a marker locus and a second locus display an inter-locus recombination frequency of 10% or less, preferably about 9% or less, still more preferably about 8% or less, yet more preferably about 7% or less, still more preferably about 6% or less, yet more preferably about 5% or less, still more preferably about 4% or less, yet more preferably about 3% or less, and still more preferably about 2% or less. In highly preferred embodiments, the relevant loci display a recombination frequency of about 1% or less, e.g., about 0.75% or less, more preferably about 0.5% or less, or yet more preferably about 0.25% or less. Two loci that are localized to the same chromosome, and at such a distance that recombination between the two loci occurs at a frequency of less than 10 (e.g., about 9%, 8%, 7%, 6%, 5%, 4%, 3%, 2%, 1%, 0.75%, 0.5%, 0.25%, or less) are also said to be “proximal to” each other. Since one cM is the distance between two markers that show a 1% recombination frequency, any marker is closely linked (genetically and physically) to any other marker that is in close proximity, e.g., at or less than 10 cM distant. Two closely linked markers on the same chromosome can be positioned 9, 8, 7, 6, 5, 4, 3, 2, 1, 0.75, 0.5 or 0.25 cM or less from each other.

The term “linkage disequilibrium” refers to a non-random segregation of genetic loci or traits for both). In either case, linkage disequilibrium implies that the relevant loci are within sufficient physical proximity along a length of a chromosome so that they segregate together with greater than random (i.e., non-random) frequency (in the case of co-segregating traits, the loci that underlie the traits are in sufficient proximity to each other). Markers that show linkage disequilibrium are considered linked. Linked loci co-segregate more than 50% of the time, e.g., from about 51% to about 100% of the time. In other words, two markers that co-segregate have a recombination frequency of less than 50% (and by definition, are separated by less than 50 cM on the same chromosome.) As used herein, linkage can be between two markers, or alternatively between a marker and a phenotype. A marker locus can be “associated with” (linked to) a trait, e.g., increased fructan/arabinoxylan. The degree of linkage of a molecular marker to a phenotypic trait is measured, e.g. as a statistical probability of co-segregation of that molecular marker with the phenotype.

Linkage disequilibrium is most commonly assessed using the measure r2, which is calculated using the formula described by Hill, W. G. and Robertson, A., Theor Appl. Genet 38:226-231 (1988). When r2=1, complete LD exists between the two marker loci, meaning that the markers have not been separated by recombination and have the same allele frequency. Values for r2 above ⅓ indicate sufficiently strong LD to be useful for mapping (Ardlie at al., Nature Reviews Genetics 3:299-309 (2002)). Hence, alleles are in linkage disequilibrium when r2 values between pairwise marker loci are greater than or equal to 0.33, 0.4, 0.5, 0.6, 0.7, 0.8, 0.9, or 1.0.

As used herein, “linkage equilibrium” describes a situation where two markers independently segregate, i.e., sort among progeny randomly. Markers that show linkage equilibrium are considered unlinked (whether or not they lie on the same chromosome).

The term “lodge” is synonymous with break. Hence, stalks that lodge are those that break at a position along the stalk.

The “logarithm of odds (LOD) value” or “LOD score” (Risch, Science 255:803-804 (1992)) is used in interval mapping to describe the degree of linkage between two marker loci. A LOD score of three between two markers indicates that linkage is 1000 times more likely than no linkage, while a LOD score of two indicates that linkage is 100 times more likely than no linkage. LOD scores greater than or equal to two may be used to detect linkage.

A “locus” is a position on a chromosome where a gene or marker is located.

“Wheat” refers to a domesticated plant of the Triticum genus, e.g., Triticum aestivus (bread wheat) or Triticum durum.

The term “wheat plant” includes: whole wheat plants, wheat plant cells, wheat plant protoplast, wheat plant cell or wheat tissue cultures from which wheat plants can be regenerated, wheat plant calli, and wheat plant cells that are intact in wheat plants or parts of wheat plants, such as wheat seeds, wheat florets, wheat germ, wheat bran, wheat endosperm, wheat cotyledons, wheat shoots, wheat stems, wheat spikelets, wheat roots, wheat root tips, and the like.

A “marker” is a nucleotide sequence or encoded product thereof (e.g., a protein) used as a point of reference. For markers to be useful at detecting recombinations, they need to detect differences, or polymorphisms, within the population being monitored. For molecular markers, this means differences at the DNA level due to polynucleotide sequence differences (e.g. SSRs, RFLPs, FLPs, SNPs). The genomic variability can be of any origin, for example, insertions, deletions, duplications, repetitive elements, point mutations, recombination events, or the presence and sequence of transposable elements. Molecular markers can be derived from genomic or expressed nucleic acids (e.g., ESTs) and can also refer to nucleic acids used as probes or primer pairs capable of amplifying sequence fragments via the use of PCR-based methods. A large number of wheat molecular markers are known in the art, and are published or available from various sources, such as the web-based Triticeae Toolbox (T3) Wheat toolbox (part of the Triticeae Coordinated Agricultural Project (T-CAP), funded by the National Institute for Food and Agriculture (NIFA) of the United States Department of Agriculture (USDA)) and the PolyMarker automated bioinformatics pipeline for SNP assay development, which uses target SNP sequence information and the IWGSC. Ramirez-Gonzalez et al, 2014, Plant Biotechnol. J. 13(5): 613-24; Ramirez-Gonzalez, et al, Bioinformatics, 31(12): 2038-39.

Markers corresponding to genetic polymorphisms between members of a population can be detected by methods well-established in the art. These include, e.g., DNA sequencing, PCR-based sequence specific amplification methods, detection of restriction fragment length polymorphisms (RFLP), detection of isozyme markers, detection of polynucleotide polymorphisms by allele specific hybridization (ASH), detection of amplified variable sequences of the plant genome, detection of self-sustained sequence replication, detection of simple sequence repeats (SSRs), detection of single nucleotide polymorphisms (SNPs), or detection of amplified fragment length polymorphisms (AFLPs). Well established methods are also known for the detection of expressed sequence tags (ESTs) and SSR markers derived from EST sequences and randomly amplified polymorphic DNA (RAPD).

A “marker allele”, alternatively an “allele of a marker locus”, can refer to one of a plurality of polymorphic nucleotide sequences found at a marker locus in a population that is polymorphic for the marker locus.

“Marker assisted selection” (or MAS) is a process by which phenotypes are selected based on marker genotypes.

“Marker assisted counter-selection” is a process by which marker genotypes are used to identify plants that will not be selected, allowing them to be removed from a breeding program or planting.

A “marker locus” is a specific chromosome location in the genome of a species when a specific marker can be found. A marker locus can be used to track the presence of a second linked locus, e.g., a linked locus that encodes or contributes to expression of a phenotypic trait. For example, a marker locus can be used to monitor segregation of alleles at a locus, such as a QTL or single gene, that are genetically or physically linked to the marker locus.

A “marker probe” is a nucleic add sequence or molecule that can be used to identify the presence of a marker locus, e.g., a nucleic acid probe that is complementary to a marker locus sequence, through nucleic add hybridization. Marker probes comprising 30 or more contiguous nucleotides of the marker locus (“all or a portion” of the marker locus sequence) may be used for nucleic acid hybridization. Alternatively, in some aspects, a marker probe refers to a probe of any type that is able to distinguish (i.e. genotype) the particular allele that is present at a marker locus.

The term “molecular marker” may be used to refer to a genetic marker, as defined above, or an encoded product thereof (e.g., a protein) used as a point of reference when identifying a linked locus. A marker can be derived from genomic nucleotide sequences or from expressed nucleotide sequences (e.g., from a spliced RNA, a cDNA, etc.), or from an encoded polypeptide. The term also refers to nucleic acid sequences complementary to or flanking the marker sequences, such as nucleic acids used as probes or primer pairs capable of amplifying the marker sequence. A “molecular marker probe” is a nucleic acid sequence or molecule that can be used to identify the presence of a marker locus, e.g., a nucleic acid probe that is complementary to a marker locus sequence. Alternatively, in some aspects, a marker probe refers to a probe of any type that is able to distinguish (i.e., genotype) the particular allele that is present at a marker locus. Nucleic acids are “complementary” when they specifically hybridize in solution, e.g., according to Watson-Crick base pairing rules. Some of the markers described herein are also referred to as hybridization markers when located on an indel region, such as the non-collinear region described herein. This is because the insertion region is, by definition, a polymorphism vis-a-vis a plant without the insertion. Thus, the marker need only indicate whether the indel region is present or absent. Any suitable marker detection technology may be used to identify such a hybridization marker, e.g., SNP technology is used in the examples provided herein.

“Nucleotide sequence”, “polynucleotide”, “nucleic acid sequence”, and “nucleic acid fragment” are used interchangeably and refer to a polymer of RNA or DNA that is single- or double-stranded, optionally containing synthetic, non-natural or altered nucleotide bases. A “nucleotide” is a monomeric unit from which DNA or RNA polymers are constructed, and consists of a purine or pyrimidine base, a pentose, and a phosphoric acid group. Nucleotides (usually found in their 5′-monophosphate form) are referred to by their single letter designation as follows: “A” for adenylate or deoxyadenylate (for RNA or DNA, respectively), “C” for cytidylate or deoxycytidylate. “G” for guanylate or deoxyguanylate. “U” for uridylate, “T” for deoxythymidylate, “R” for purines (A or G), “Y” for pyrimidines (C or T), “K” for G or T, “H” for A or C or T, “I” for inosine, and “N” for any nucleotide.

The terms “phenotype”, or “phenotypic trait” or “trait” refers to one or more traits of an organism. The phenotype can be observable to the naked eye, or by any other means of evaluation known in the art, e.g., microscopy, biochemical analysis, or an electromechanical assay. In some cases, a phenotype is directly controlled by a single gene or genetic locus, i.e., a “single gene trait”. In other cases, a phenotype is the result of several genes.

A “physical map” of the genome is a map showing the linear order of identifiable landmarks (including genes, markers, etc.) on chromosome DNA. However, in contrast to genetic maps, the distances between landmarks are absolute (for example, measured in base pairs or isolated and overlapping contiguous genetic fragments) and not based on genetic recombination.

A “plant” can be a whole plant, any part thereof, or a cell or tissue culture derived from a plant. Thus, the term “plant” can refer to any of: whole plants, plant components or organs (e.g., leaves, stems, roots, etc.), plant tissues, seeds, plant cells, and/or progeny of the same. A plant cell is a cell of a plant, taken from a plant, or derived through culture from a cell taken from a plant.

A “polymorphism” is a variation in the DNA that is too common to be due merely to new mutation. A polymorphism must have a frequency of at least 1% in a population. A polymorphism can be a single nucleotide polymorphism, or SNP, or an insertion/deletion polymorphism, also referred to herein as an “indel”.

The “probability value” or “p-value” is the statistical likelihood that the particular combination of a phenotype and the presence or absence of a particular marker allele is random. Thus, the lower the probability score, the greater the likelihood that a phenotype and a particular marker will co-segregate. In some aspects, the probability score is considered “significant” or “nonsignificant”. In some embodiments, a probability score of 0.05 (p=0.05, or a 5% probability) of random assortment is considered a significant indication of co-segregation. However, an acceptable probability can be any probability of less than 50% (p=0.5). For example, a significant probability can be less than 0.25, less than 0.20, less than 0.15, less than 0.1, less than 0.05, less than 0.01, or less than 0.001.

The term “progeny” refers to the offspring generated from a cross.

A “progeny plant” is generated from a cross between two plants.

A “reference sequence” is a defined sequence used as a basis for sequence comparison. The reference sequence is obtained by genotyping a number of lines at the locus, aligning the nucleotide sequences in a sequence alignment program (e.g. SEQUENCHER), and then obtaining the consensus sequence of the alignment.

A “single nucleotide polymorphism (SNP)” is a DNA sequence variation occurring when a single nucleotide—A, T, C or G—in the genome (or other shared sequence) differs between members of a biological species or paired chromosomes in an individual. For example, two sequenced DNA fragments from different individuals, AAGCCTA to AAGCTTA, contain a difference in a single nucleotide.

A “topeross test” is a progeny test derived by crossing each parent with the same tester, usually a homozygous line. The parent being tested can be an open-pollinated variety, a cross, or an inbred line.

The phrase “under stringent conditions” refers to conditions under which a probe or polynucleotide will hybridize to a specific nucleic acid sequence, typically in a complex mixture of nucleic acids, but to essentially no other sequences. Stringent conditions are sequence-dependent and will be different in different circumstances.

Longer sequences hybridize specifically at higher temperatures. Generally, stringent conditions are selected to be about 5-10° C. lower than the thermal melting point (Tm) for the specific sequence at a defined ionic strength pH. The Tm is the temperature (under defined ionic strength, pH, and nucleic acid concentration) at which 50% of the probes complementary to the target hybridize to the target sequence at equilibrium (as the target sequences are present in excess, at Tm, 50 of the probes are occupied at equilibrium). Stringent conditions will be those in which the salt concentration is less than about 1.0 M sodium ion, typically about 0.01 to 1.0 M sodium on concentration (or other salts) at pH 7.0 to 8.3, and the temperature is at least about 30° C. for short probes (e.g., 10 to 50 nucleotides) and at least about 60° C. for long probes (e.g. greater than 50 nucleotides). Stringent conditions may also be achieved with the addition of destabilizing agents such as form amide. For selective or specific hybridization, a positive signal is at least two times background, preferably 10 times background hybridization. Exemplary stringent hybridization conditions are often: 50% formamide, 5×SSC, and 1% SDS, incubating at 42° C., or, 5×SSC, 1% SOS, incubating at 65° C., with wash in 0.2×SSC, and 0.1% SDS at 65° C. For PCR, a temperature of about 36° C. is typical for low stringency amplification, although annealing temperatures may vary between about 32° C. and 48° C., depending on primer length. Additional guidelines for determining hybridization parameters are provided in numerous references.

Sequence alignments and percent identity calculations may be determined using a variety of comparison methods designed to detect homologous sequences including, but not limited to, the MEGALIGN® program of the LASERGENE® bioinformatics computing suite (DNASTAR® Inc., Madison, Wis.). Unless stated otherwise, multiple alignment of the sequences provided herein were performed using the Clustal V method of alignment (Higgins and Sharp, CABIOS. 5:151-153 (1989)) with the default parameters (GAP PENALTY=10, GAP LENGTH PENALTY=10), Default parameters for pairwise alignments and calculation of percent identity of protein sequences using the Clustal V method are KTUPLE=1, GAP PENALTY=3, WINDOW=5 and DIAGONALS SAVED=5. For nucleic adds these parameters are KTUPLE=2, GAP PENALTY=5, WINDOW=4 and DIAGONALS SAVED=4. After alignment of the sequences, using the Clustal V program, it is possible to obtain “percent identity” and “divergence” values by viewing the “sequence distances” table on the same program; unless stated otherwise, percent identities and divergences provided and claimed herein were calculated in this manner.

Before describing the present invention in detail, it should be understood that this invention is not limited to particular embodiments. It also should be understood that the terminology used herein is for the purpose of describing particular embodiments, and is not intended to be limiting. As used herein and in the appended claims, terms in the singular and the singular forms “a”, “an” and “the”, for example, include plural referents unless the content clearly dictates otherwise. Thus, for example, reference to “plant”, “the plant” or “a plant” also includes a plurality of plants. Depending on the context, use of the term “plant” can also include genetically similar or identical progeny of that plant. The use of the term “a nucleic acid” optionally includes many copies of that nucleic acid molecule.

Genetic Mapping

It has been recognized for quite some time that specific genetic loci correlating with particular phenotypes can be mapped in an organism's genome. The plant breeder can advantageously use molecular markers to identify desired individuals by detecting marker alleles that show a statistically significant probability of co-segregation with a desired phenotype, manifested as linkage disequilibrium. By identifying a molecular marker or clusters of molecular markers that co-segregate with a trait of interest, the breeder is able to rapidly select a desired phenotype by selecting for the proper molecular marker allele (a process called marker-assisted selection, or MAS).

A variety of methods well known in the art are available for detecting molecular markers or clusters of molecular markers that co-segregate with a trait of interest, such as the QTLs for increased fructan/arabinoxylan disclosed herein. Generally, these methods involve the detection of markers, for which alternative genotypes (or alleles) have significantly different average phenotypes. Thus, marker loci are compared to determine the magnitude of difference among alternative genotypes (or alleles) or the level of significance of that difference. Trait genes are inferred to be located nearest the marker(s) that have the greatest associated genotypic difference.

Two such methods used to detect trait loci of interest are: 1) Population-based association analysis and 2) Traditional linkage analysis. In a population-based association analysis, lines are obtained from pre-existing populations with multiple founders, e.g. elite breeding lines. Population-based association analyses rely on the decay of linkage disequilibrium (LD) and the idea that in an unstructured population, only correlations between genes controlling a trait of interest and markers closely linked to those genes will remain after so many generations of random mating. In reality, most pre-existing populations have population substructure. Thus, the use of a structured association approach helps to control population structure by allocating individuals to populations using data obtained from markers randomly distributed across the genome, thereby minimizing disequilibrium due to population structure within the individual populations (also called subpopulations). The phenotypic values are compared to the genotypes (alleles) at each, marker locus for each line in the subpopulation. A significant marker-trait association indicates the dose proximity between the marker locus and one or more genetic loci that are involved in the expression of that trait.

The same principles underlie traditional linkage analysis; however, LD is generated by creating a population from a small number of founders. The founders are selected to maximize the level of polymorphism within the constructed population, and polymorphic sites are assessed for their level of cosegregation with a given phenotype. A number of statistical methods have been used to identify significant marker-trait associations. One such method is an interval mapping approach (Lander and Botstein, Genetics 121:185-199 (1989), in which each of many positions along a genetic map (say at 1 cM intervals) is tested for the likelihood that a gene controlling a trait of interest is located at that position. The genotype/phenotype data are used to calculate for each test position a LOD score (log of likelihood ratio). When the LOD score exceeds a threshold value, there is significant evidence for the location of a gene controlling the trait of interest at that position on the genetic map (which will fall between two particular marker loci).

Markers Associated with Increased Fructan/Arabinoxylan

Markers associated with increased fructan/arabinoxylan are identified herein. The methods of the invention involve detecting the presence of at least one marker allele associated with the enhanced fructan/arabinoxylan content in a wheat plant. The marker locus can be selected from any of the marker loci provided in Tables 1, 2, and 3, including markers for haplotypes, and any other marker linked to these markers. Linked markers can be determined by reference to resources such as the Triticeae Toolbox (T3) Wheat toolbox (part of the Triticeae Coordinated Agricultural Project (T-CAP), funded by the National Institute for Food and Agriculture (NIFA) of the United States Department of Agriculture (USDA)) and the PolyMarker automated bioinformatics pipeline for SNP assay development, which uses target SNP sequence information and the IWGSC. See Ramirez-Gonzalez et al, 2014, Plant Biotechnol. J. 13(5): 613-24; Ramirez-Gonzalez, et al, Bioinformatics, 31(12): 2038-39. Methods known in the art can be used to (i) establish the presence or absence of particular markers for the QTLs disclosed herein in a reference population and (ii) screen for the presence or absence of the markers corresponding to one or more QTL disclosed herein for increased fructan/arabinoxylan.

Marker loci associated with increased fructan/arabinoxylan can include any polynucleotide that binds to (or otherwise indicates the presence of) contiguous DNA between and including the left and right interval markers for one or more of QTL 1A, QTL 1B, QTL 2B-1, QTL 2B-2, QTL 2D, QTL 6B, QTL 7A-1, QTL 7A-2, and QTL 7B, optionally in combination with the left and right interval markers for QTL 7A-3.

A common measure of linkage is the frequency with which traits cosegregate. This can be expressed as a percentage of cosegregation (recombination frequency) or in centiMorgans (cM). The cM is a unit of measure of genetic recombination frequency. One cM is equal to a 1% chance that a trait at one genetic locus will be separated from a trait at another locus due to crossing over in a single generation (meaning the traits segregate together 99% of the time). Because chromosomal distance is approximately proportional to the frequency of crossing over events between traits, there is an approximate physical distance that correlates with recombination frequency.

Marker loci are themselves traits and can be assessed according to standard linkage analysis by tracking the marker loci during segregation. Thus, one cM is equal to a 1% chance that a marker locus will be separated from another locus, due to crossing over in a single generation.

Other markers linked to the markers listed in Table 2 can be used to predict increased fructan/arabinoxylan in a wheat plant. This includes any marker within 50 cM of, the markers associated with the left and right interval markers for one or more of QTL 1A, QTL 1B, QTL 2B-1, QTL 2B-2, QTL 2D, QTL 6B, QTL 7A-1, QTL 7A-2, and QTL 7B, which are associated with increased fructan/arabinoxylan. The closer a marker is to a gene controlling a trait of interest, the more effective and advantageous that marker is as an indicator for the desired trait. Closely linked loci display an inter-locus cross-over frequency of about 10% or less, preferably about 9% or less, still more preferably about 8% or less, yet more preferably about 7% or less, still more preferably about 6% or less, yet more preferably about 5% or less, still more preferably about 4% or less, yet more preferably about 3% or less, and still more preferably about 2% or less. In highly preferred embodiments, the relevant loci (e.g., a marker locus and a target locus) display a recombination frequency of about 1% or less, e.g., about 0.75% or less, more preferably about 0.5% or less, or yet more preferably about 0.25% or less. Thus, the loci are about 10 cM, 9 cM, 8 cM, 7 cM, 6 cM, 5 cM, 4 cM, 3 cM, 2 cM, 1 cM, 0.75 cM, 0.5 cM or 0.25 cM or less apart. Put another way, two loci that are localized to the same chromosome, and at such a distance that recombination between the two loci occurs at a frequency of less than 10% (e.g., about 9%, 8% 7%, 6%, 5%, 4%, 3%, 2% 1%, 0.75%, 0.5%, 0.25.degree., or less) are said to be “proximal to” each other.

Although particular marker alleles can show co-segregation with increased fructan/arabinoxylan, it is important to note that the marker locus is not necessarily responsible for the expression of the increased fructan/arabinoxylan phenotype. For example, it is not a requirement that the marker polynucleotide sequence be part of a gene that imparts increased fructan/arabinoxylan (for example, be part of the gene open reading frame). The association between a specific marker allele and the increased fructan/arabinoxylan phenotype is due to the original “coupling” linkage phase between the marker allele and the allele in the ancestral wheat line from which the allele originated. Eventually, with repeated recombination, crossing over events between the marker and genetic locus can change this orientation. For this reason, the favorable marker allele may change depending on the linkage phase that exists within the resistant parent used to create segregating populations. This does not change the fact that the marker can be used to monitor segregation of the phenotype. It only changes which marker allele is considered favorable in a given segregating population.

The term “chromosomal interval” designates any and all intervals defined by any of the markers set forth in this invention. Several chromosomal interval that correlate with increased fructan/arabinoxylan are provided by the invention. For example, the invention provides the intervals for QTL 1A, QTL 1B, QTL 2B-1, QTL 2B-2, QTL 2D, QTL 6B, QTL 7A-1, QTL 7A-2, and QTL 7B, comprises and flanked by the corresponding left right interval markers identified in Table 1, above. These intervals, including combination thereof can be combined with intervals for 7A-3 described herein

A variety of methods well known in the art are available for identifying chromosomal intervals. The boundaries of such chromosomal intervals are drawn to encompass markers that will be linked to the gene controlling the trait of interest. In other words, the chromosomal interval is drawn such that any marker that lies within that interval (including the terminal markers that define the boundaries of the interval) can be used as a marker for increased fructan/arabinoxylan. The interval described above encompasses a cluster of markers that co-segregate with increased fructan/arabinoxylan. The clustering of markers occurs in relatively small domains on the chromosomes, indicating the presence of a gene controlling the trait of interest in those chromosome regions. The interval was drawn to encompass the markers that co-segregate with increased fructan/arabinoxylan. The interval encompasses markers that map within the interval as well as the markers that define the termini. An interval described by the terminal markers that define the endpoints of the interval will include the terminal markers and any marker localizing within that chromosomal domain, whether those markers are currently known or unknown.

Chromosomal intervals can also be defined by markers that are linked to (show linkage disequilibrium with) a marker of interest, and is a common measure of linkage disequilibrium (LD) in the context of association studies. If the r² value of LD between any chromosome marker locus lying within the indicated left and right intervals in Table 1 (or any other subinterval within these intervals) and an identified marker within that interval that has an allele associated with increased fructan/arabinoxylan is greater than ⅓ (Ardlie et al. Nature Reviews Genetics 3:299-309 (2002)), the loci are linked.

A marker of the invention can also be a combination of alleles at marker loci, otherwise known as a haplotype. The skilled artisan would expect that there might be additional polymorphic sites at marker loci in and around the markers identified herein, wherein one, or more polymorphic sites is in linkage disequilibrium (LD) with an allele associated with increased fructan/arabinoxylan. Two particular alleles at different polymorphic sites are said to be in LD if the presence of the allele at one of the sites tends to predict the presence of the allele at the other site on the same chromosome (Stevens, Mol. Diag. 4:309-17 (1999)).

Marker Assisted Selection

Molecular markers can be used in a variety of plant breeding applications (e.g. see Staub et al. (1996) Hortscience 729-741; Tanksley (1983) Plant Molecular Biology Reporter 1: 3-8). One of the main areas of interest is to increase the efficiency of backcros sing and introgressing genes using marker-assisted selection (MAS). A molecular marker that demonstrates linkage with a locus affecting a desired phenotypic trait provides a useful tool for the selection of the trait in a plant population. This is particularly true where the phenotype is hard to assay, e.g. many disease resistance traits, or, occurs at a late stage in plant development, e.g. kernel characteristics. Since DNA marker assays are less laborious and take up less physical space than field phenotyping, much larger populations can be assayed, increasing the chances of finding a recombinant with the target segment from the donor line moved to the recipient line. The closer the linkage, the more useful the marker, as recombination is less likely to occur between the marker and the gene causing the trait, which can result in false positives. Having flanking markers decreases the chances that false positive selection will occur as a double recombination event would be needed. The ideal situation is to have a marker in the gene itself, so that recombination cannot occur between the marker and the gene. Such a marker is called a ‘perfect marker’.

When a gene is introgressed by MAS, it is not only the gene that is introduced but also the flanking regions (Gepts. (2002). Crop Sci.; 42: 1780-1790). This is referred to as “linkage drag.” In the case where the donor plant is highly unrelated to the recipient plant, these flanking regions carry additional genes that may code for agronomically undesirable traits. This “linkage drag” may also result in reduced yield or other negative agronomic characteristics even after multiple cycles of backcrossing into the elite wheat line. This is also sometimes referred to as “yield drag.” The size of the flanking region can be decreased by additional backcrossing, although this is not always successful, as breeders do not have control over the size of the region or the recombination breakpoints (Young et al, (1998) Genetics 120:579-585). In classical breeding it is usually only by chance that recombinations are selected that contribute to a reduction in the size of the donor segment (Tanksley et al. (1989). Biotechnology 7: 257-264). Even after 20 backcrosses in backcrosses of this type, one may expect to find a sizable piece of the donor chromosome still linked to the gene being selected. With markers however, it is possible to select those rare individuals that have experienced recombination near the gene of interest. In 150 backcross plants, there is a 95% chance that at least one plant will have experienced a crossover within 1 cM of the gene, based on a single meiosis map distance. Markers will avow unequivocal identification of those individuals. With one additional backcross of 300 plants, there would be a 95% chance of a crossover within 1 cM single meiosis map distance of the other side of the gene, generating a segment around the target gene of less than 2 cM based on a single meiosis map distance. This can be accomplished in two generations with markers, while it would have required on average 100 generations without markers (See Tanksley et al., supra). When the exact location of a gene is known, flanking markers surrounding the gene can be utilized to select for recombinations in different population sizes. For example, in smaller population sizes, recombinations may be expected further away from the gene, so more distal flanking markers would be required to detect the recombination.

The availability of the wheat reference genome, and the integrated linkage maps of the wheat genome containing increasing densities of public wheat markers, has facilitated wheat genetic mapping and MAS. See, e.g. Triticeae Toolbox (T3) Wheat toolbox (part of the Triticeae Coordinated Agricultural Project (T-CAP), funded by the National Institute for Food and Agriculture (NIFA) of the United States Department of Agriculture (USDA)) and the PolyMarker automated bioinformatics pipeline for SNP assay development, which uses target SNP sequence information and the IWGSC. Ramirez-Gonzalez et al, 2014, Plant Biotechnol. J. 13(5): 613-24; Ramirez-Gonzalez, et al, Bioinformatics, 31(12): 2038-39.

The key components to the implementation of MAS are (i) defining the population within which the marker-trait association will be determined, which can be a segregating population, or a random or structured population; (ii) monitoring the segregation or association of polymorphic markers relative to the trait, and determining linkage or association using statistical methods; (iii) defining a set of desirable markers based on the results of the statistical analysis, and (iv) the use and/or extrapolation of this information to the current set of breeding germplasm to enable marker-based selection decisions to be made. The markers described in this disclosure, as well as other marker types such as SSRs and FLPs, can be used in marker assisted selection protocols.

SSRs can be defined as relatively short runs of tandem repeated DNA with lengths of 6 bp or less (Tautz (1989) Nucleic Acid Research 17: 6463-6471; Wang et al. (1994) Theoretical and Applied Genetics, 88:1-6) Polymorphisms arise due to variation in the number of repeat units, probably caused by slippage during DNA replication (Levinson and Gutman (1987) Mol. Biol. Evol. 4: 203-221). The variation in repeat length may be detected by designing PCR primers to the conserved non-repetitive flanking regions (Weber and May (1989) Am. J. Hum. Genet. 44:388-396), SSRs are highly suited to mapping and MAS as they are multi-allelic, codominant, reproducible and amenable to high throughput automation (Rafalski et al. (1996) Generating and using DNA markers in plants. In Non-mammalian genomic analysis: a practical guide. Academic Press, pp 75-135).

Various types of SSR markers can be generated, and SSR profiles from resistant lines can be obtained by gel electrophoresis of the amplification products. Scoring of marker genotype is based on the size of the amplified fragment.

Various types of FLP markers can also be generated. Most commonly, amplification primers are used to generate fragment length polymorphisms. Such FLP markers are in many ways similar to SSR markers, except that the region amplified by the primers is not typically a highly repetitive region. Still, the amplified region, or amplicon, will have sufficient variability among germplasm, often due to insertions or deletions, such that the fragments generated by the amplification primers can be distinguished among polymorphic individuals, and such indels are known to occur in wheat.

SNP markers detect single base pair nucleotide substitutions. Of all the molecular marker types, SNPs are the most abundant, thus having the potential to provide the highest genetic map resolution (Bhattramakki et al. 2002 Plant Mol. Biol. 48:539-547). SNPs can be assayed at an even higher level of throughput than SSRs, in a so-called ‘ultra-high-throughput’ fashion, as they do not require large amounts of DNA and automation of the assay may be straight-forward. SNPs also have the promise of being relatively low-cost systems. These three factors together make SNPs highly attractive for use in MAS. Several methods are available for SNP genotyping, including but not limited to, hybridization, primer extension, oligonucleotide ligation, nuclease cleavage, mini-sequencing and coded spheres. Such methods have been reviewed in: Gut (2001) Hum. Mutat. 17 pp, 475-492: Shi (2001) Clin. Chem. 47, pp. 164-172; Kwok (2000) Pharmacogenomics 1, pp. 95-100: Bhattramakki and Rafalski (2001) Discovery and application of single nucleotide polymorphism markers in plants. In: R, J Henry, Ed, Plant Genotyping: The DNA Fingerprinting of Plants, CABI Publishing, V. Vallingford. A wide range of commercially available technologies utilize these and other methods to interrogate SNPs including Masscode™. (Qiagen), Invader® (Third Wave Technologies), SnapShot® (Applied Biosystems), Taqman® (Applied Biosystems) and Beadarrays™ (Illumina).

A number of SNPs together within a sequence, or across linked sequences, can be used to describe a haplotype for any particular genotype (Ching et al. (2002), BMC Genet. 3:19 pp Gupta et al. 2001, Rafalski (2002b), Plant Science 162:329-333). Haplotypes can be more informative than single SNPs and can be more descriptive of any particular genotype. For example, single SNP may be allele ‘T’ for a specific line or variety with increased fructan/arabinoxylan, but the allele ‘T’ might also occur in the wheat breeding population being utilized for recurrent parents. In this case, a haplotype, e.g. a combination of alleles at linked SNP markers, may be more informative. Once a unique haplotype has been assigned to a donor chromosomal region, that haplotype can be used in that population or any subset thereof to determine whether an individual has a particular gene. See, for example, WO2003054229. Using automated high throughput marker detection platforms known to those of ordinary skill in the art makes this process highly efficient and effective.

The markers listed in Tables 1, 2 and 3 can be readily used to obtain additional polymorphic SNPs (and other markers) within the QTL interval listed in this disclosure. Markers within the described map region can be hybridized to BACs or other genomic libraries, or electronically aligned with genome sequences, to find new sequences in the same approximate location as the described markers.

In addition to SSR's, FLPs and SNPs, as described above, other types of molecular markers are also widely used, including but not limited to expressed sequence tags (ESTs), SSR markers derived from EST sequences, randomly amplified polymorphic DNA (RAPD), and other nucleic acid based markers.

Isozyme profiles and linked morphological characteristics can, in some cases, also be indirectly used as markers. Even though they do not directly detect DNA differences, they are often influenced by specific genetic differences. However, markers that detect DNA variation are far more numerous and polymorphic than isozyme or morphological markers (Tanksley (1983) Plant Molecular Biology Reporter 1:3-8).

Sequence alignments or contigs may also be used to find sequences upstream or downstream of the specific markers listed herein. These new sequences, close to the markers described herein, are then used to discover and develop functionally equivalent markers. For example, different physical and/or genetic maps are aligned to locate equivalent markers not described within this disclosure but that are within similar regions. These maps may be within the wheat species, or even across other species that have been genetically or physically aligned with wheat, such as rice, wheat, barley or sorghum.

In general, MAS uses polymorphic markers that have been identified as having a significant likelihood of co-segregation with increased fructan/arabinoxylan. Such markers are presumed to map near a gene or genes that give the plant its increased fructan/arabinoxylan phenotype, and are considered indicators for the desired trait, or markers. Plants are tested for the presence of a desired allele in the marker, and plants containing a desired genotype at one or more loci are expected to transfer the desired genotype, along with a desired phenotype, to their progeny. The means to identify wheat plants that have increased fructan/arabinoxylan by identifying plants that have a specified allele at any one of marker loci described herein, including QTL 1A, QTL 1B, QTL 2B-1, QTL 2B-2, QTL 2D, QTL 6B, QTL 7A-1, QTL 7A-2, and QTL 7B are presented herein.

The QTL intervals presented herein finds use in MAS to select plants that demonstrate increased fructan/arabinoxylan. Any marker that maps within one (or a combination) of the chromosome intervals defined by and including the left and right intervals in Table 1 can be used for this purpose. In addition, haplotypes comprising alleles at one or more marker loci within the QTL 1A, QTL 1B, QTL 2B-1, QTL 2B-2, QTL 2D, QTL 6B, QTL 7A-1, QTL 7A-2, and QTL 7B intervals defined by and including the left and right intervals in Table 1 can be used to introduce increased fructan/arabinoxylan into wheat lines or varieties. Any allele or haplotype that is in linkage disequilibrium with an allele associated with increased fructan/arabinoxylan can be used in MAS to select plants with increased fructan/arabinoxylan.

The following examples are offered to illustrate, but not to limit, the appended claims. It is understood that the examples and embodiments described herein are for illustrative purposes only and that persons skilled in the art will recognize various reagents or parameters that can be altered without departing from the spirit of the invention or the scope of the appended claims.

### EXAMPLE 1

A four-way multiparent advanced generation inter-cross (“MAGIC”) population has been described in Huang et al., 2012 Plant Biotech J 10: 826-39. Four elite bread wheat cultivars (Yitpi, Chara, Baxter, and Westonia) were intercrossed in a set mating design to combine the genomes of all four parental cultivars. Multiple generations of crossings produced a population of 1579 progeny, and 1162 markers were mapped in the population across all 21 chromosomes. An eight-way MAGIC population was also created using elite wheat cultivars Westonia, Yitpi, AC Barrie, Xiaoyan 54, Pastor, Alsen, Baxter, and Volcani. This was done generally along the same lines as the four-way MAGIC population and as described in Cavanaugh 2008, Current Opinion in Plant Biol. 11: 215-221.

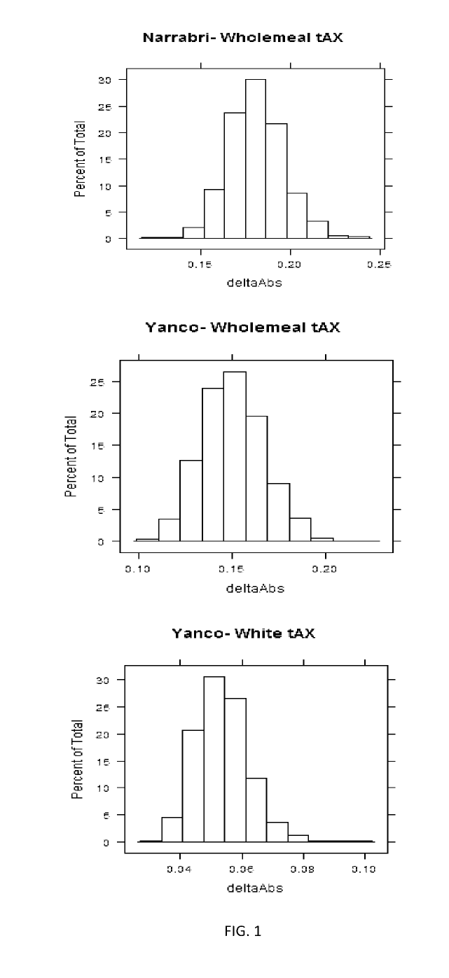

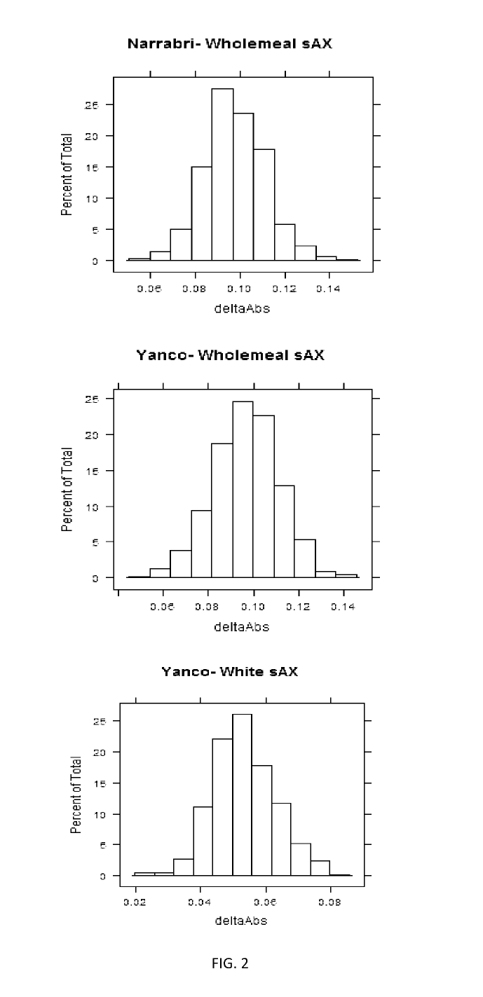

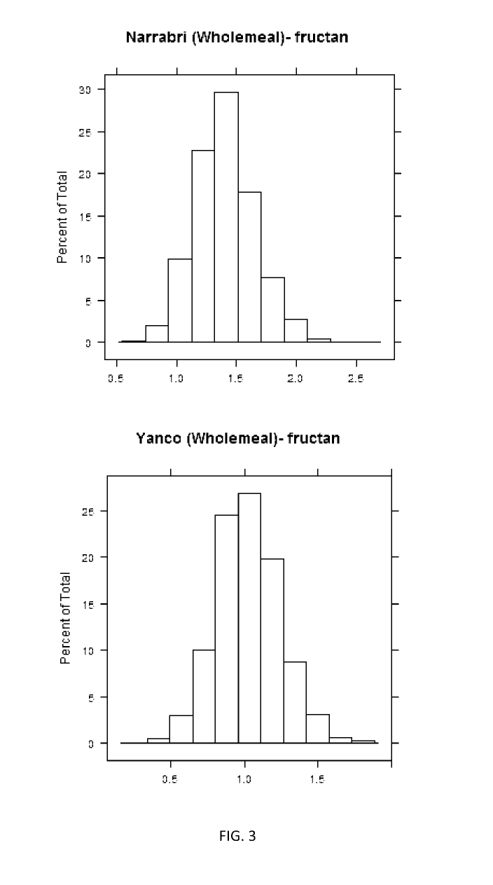

MAGIC populations were grown at different sites in New South Wales, in different years. In particular populations were grown in Yanco and Narrabi. Wholemeal and white flour samples (all samples included replicates) were evaluated for fructan, soluble arabinoxylan, and total arabinoxylan content. Fructan and arabinoxylan (soluble and total) assays revealed variation in fructan and arabinoxylan (soluble and total) content among the lines from the four-way population at different sites and years. Results are shown in FIGS. 1, 2, and 3.

Fructan and arabinoxylan content was shown to be generally heritable across the different sites and years.

### EXAMPLE 2

In view of the demonstrated variability and heritability of fructan/arabinoxylan content described in Example 1, QTL analysis was conducted using multivariate Multi-Parent Whole Genome Average Interval Mapping (MPWGAIM) approaches. See Verbyla et al., 2014a, Theor. Appl. Genet. 127:1753-70 and Verbyla et al., 2014b, G3: Genes, Genomes, Genetics 4: 1569-84. Generally, MPWGAIM (univariate and multivariate) utilizes the probability of inheriting founder alleles across the whole genome by simultaneously incorporating all information in the analysis, overcoming the need for repeated genome scans. A random effects working model is used in which all intervals are allowed to contain a possible QTL. A forward selection approach is used to select QTL. A likelihood ratio test of significance is conducted to decide if selection of a putative QTL is warranted or if selection should cease. An outlier statistic is used to select the most likely location for each QTL at the stage of the forward selection process. The approach allows for any non-genetic effects, such as experimental design terms, to be easily included in the base models.

DNA, including molecular marker genotyping DNA, was isolated from leaf material of single plants of the F6-derived RIL lines of the 4-way MAGIC population using Machery-Nagel NucleoSpin 96 Plant II kits supplied by Scientifix (Clayton, Vic., Australia). A different method was used for 8-way population.

Wheat DNA was analysed on the 90K single nucleotide polymorphism (SNP) chip array (“90K chip) disclosed by Wang et al., 2014, Plant Biotech J. 12: 787-796. SNPs were assayed using Infinium iSelect® (IIlumina, San Diego, Calif.) SNP assays, as described in Cavanaugh et al., 2013, Proc. Natl. Acad. Sci. 10(20): 8057-8062. For the 4-way population, this data was added to marker data previously described in Verbyla et al., 2014a and Verbyla et al., 2014b to create an integrated map including microsatellites (SSRs), DArTs, and 9K and 90K SNP markers. In the 8-way population, the 90K SNP data was supplemented by a set of SSRs. Both maps were constructed utilising R package mpMap. See Huang and George, 2011, Bioinformatics, 27: 727-29. Results are shown in Tables 4.

TABLE 4

| QTL | | | dist | | | | | Target |
|---|---|---|---|---|---|---|---|---|
| (Chr) | Trait | Marker | (cM) | Westonia | Chara | Yitpi | Baxter | Variety |
| 1A | sAX, | wsnp_Ex_c3572_6531810, | 110.42-115.15 | AA | AA | BB | AA | For Yitpi |
| | tAX | wsnp_Ex_c3142_5808713 | | | | | | |
| 1B | sAX, | wsnp_Ex_c10869_17671164, | 114.21 | AA | BB | AA | BB | For Baxter |
| | tAX | IAAV1364, | | | | | | or Chara |
| | | BobWhite_c4147_1351 | | | | | | |
| 2B | tAX | BobWhite_c6472_601, | 83.91-84.01 | AA | AA | AA | BB | Against |
| | | RAC875_c96504_413 | | | | | | Baxter |
| 2B | Fruc | Excalibur_c6502_397, | 158.11-160.06 | BB | AA | AA | AA | For |
| | | Excalibur_c25234_143 | | | | | | Westonia |
| 2D | Fruc | Haplotype: (i) TA012840-0369 | 55.67-55.08 | AA, AA | BB, BB | BB, BB | AA, BB | For Baxter |
| | | and (ii) one of | | | | | | |
| | | (BS00000905_51, | | | | | | |
| | | wsnp_Ex_c25311_34578436) | | | | | | |
| 6B | sAX, | wsnp_Ku_c11422_18657479, | 84.1-87.99 | AA | BB | AA | BB | for Baxter |
| | tAX | Ku_c2212_411 | | | | | | or Chara |
| 7A-1 | Fruc₁ | Excalibur_c30730_253, | 42.69-43.28 | AA | AA | BB | AA | Against |
| | | BS00065529_51 | | | | | | Yitpi |
| 7A-2 | sAX | RAC875_c32212_84, | 85.25-85.33 | BB | AA | AA | AA | For |
| | | IACX3013 | | | | | | Westonia |
| 7A-3 | Fruc₂ | BS00085135_51, | 175.19-175.25 | AA | BB | AA | BB | for Baxter |
| | | Tdurum_contig52015_ 1090 | | | | | | or Chara |
| 7B | tAX | Haplotype: | 153.2-164.54 | AA, AA, | BB, BB, | BB, AA, | BB, AA, | For Baxter |
| | | (i) one of (Ex_c101666_634, | | AA | AA | BB | AA | |
| | | wsnp_Ex_c2103_3947695) and | | | | | | |
| | | (ii) one of (GENE- | | | | | | |
| | | 4833_102, snp90kbi_Kukri_c67849_109) | | | | | | |
| | | and (iii) one of | | | | | | |
| | | (Excalibur_c40122_280, | | | | | | |
| | | snp90kbi_BobWhite_c44404_312, | | | | | | |
| | | snp90kpoly_Tdurum_contig31682_53) | | | | | | |

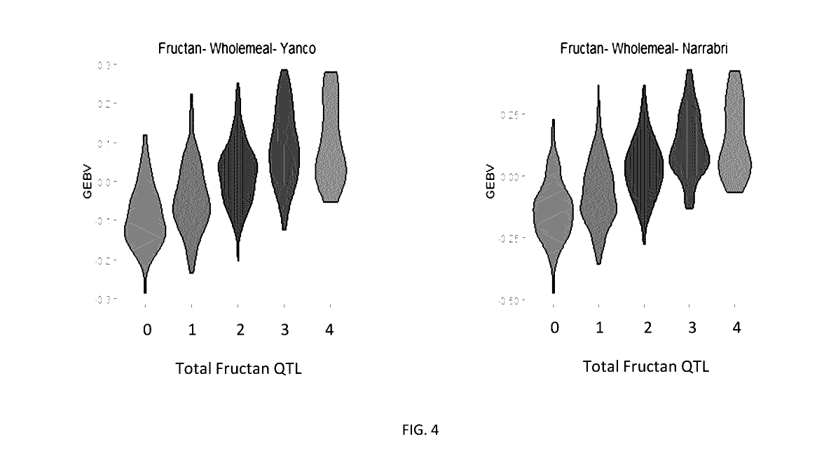

Further analysis demonstrated that the presence of multiple fructan QTL alleles had an additive effect on fructan content throughout a given population. Yanco and Narrabi MAGIC populations were analyzed for fructan/arabinoxylan content and the presence or absence of all QTLs associated with fructan disclosed by the invention. Specifically, QTLs were significantly associated with higher fructan content relative to mean fructan levels. Furthermore, relative to plants having 0 fructan QTLs, wheat plants having 1 fructan QTL disclosed herein had higher fructan content, wheat plants having 2 fructan QTLs disclosed herein had even higher fructan content. Additionally, wheat plants having 3 fructan QTLs disclosed herein had still higher fructan content than those having fewer than 3 QTLs, and wheat plants having all 4 fructan QTLs disclosed herein had highest fructan content. The additive effect of QTLs is shown in FIG. 4.

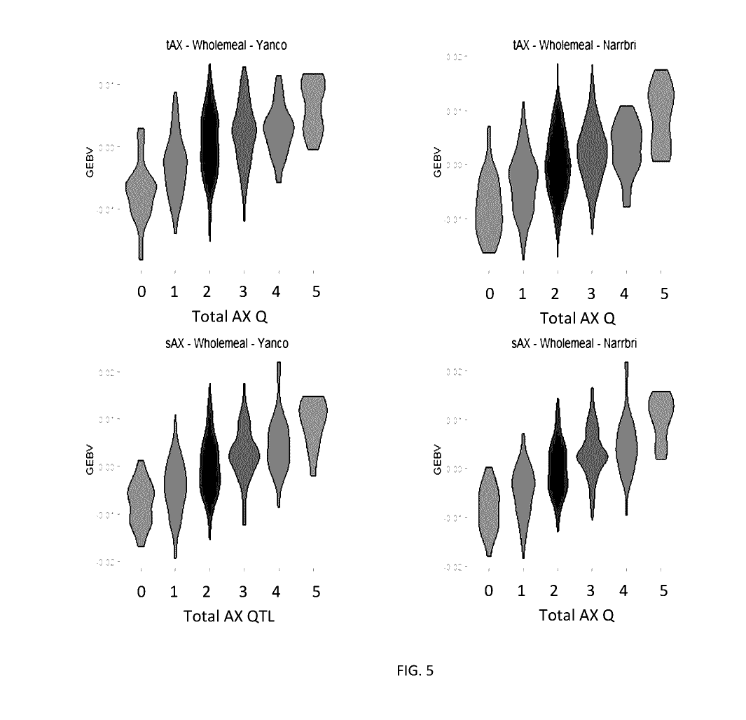

Similar analysis demonstrated that the presence of multiple arabinoxylan QTL alleles had an additive effect on arabinoxylan content throughout a given population. Yanco and Narrabi MAGIC populations were analyzed for arabinoxylan content and the presence or absence of all four QTLs associated with arabinoxylan disclosed by the invention. The results demonstrated that relative to wheat plants having 0 arabinoxylan QTLs, wheat plants having 1 arabinoxylan QTL disclosed herein had higher arabinoxylan content, and wheat plants having 2 arabinoxylan QTLs disclosed herein had even higher arabinoxylan content. Furthermore, the wheat plants having 3 arabinoxylan QTLs disclosed herein had still higher arabinoxylan content than those having fewer than 3 QTLs, wheat plants having 4 arabinoxylan QTLs had the second highest arabinoxylan content, and wheat plants having all 5 novel arabinoxylan QTLs disclosed herein had highest arabinoxylan content. The additive effects QTLs for both total arabinoxylan and soluble arabinoxylan are demonstrated in FIG. 5.

The foregoing example demonstrates that markers on QTL 1A, QTL 1B, QTL 2B-1, QTL 2B-2, QTL 2D, QTL 6B, QTL 7A-1, QTL 7A-2, and QTL 7B can be used according to the invention to identify from a population of wheat plants, individual plants having a higher probability of carrying a heritable increased fructan/arabinoxylan trait. The foregoing example also demonstrates the usefulness of combining these markers with each other and in combinations that further include QTL 7A-3, in accordance with the invention to identify individual plants having a higher probability of carrying a heritable increased fructan/arabinoxylan trait.

### EXAMPLE 3

The statistical significance of each QTL to increased fructan/arabinoxylan trait was rigorously evaluated individually and in particular combinations. The impact of each fructan and arabinoxylan QTL within MAGIC populations grown in Yanco and Narrabi are shown in Table 5.

TABLE 5

| | | Yanco | | | Narrabi | | |
|---|---|---|---|---|---|---|---|
| | | | | Significance | | | Significance |
| QTL | Trait | “+QTL” | “−QTL” | (p-value) | “+QTL” | “−QTL” | (p-value) |
| 2B | Fruc | 0.02156145 | −0.006009722 | 0.003187 | 0.04234234 | −0.01156724 | 4.81E−05 |
| 2D | Fruc | 0.03423653 | −0.01283869 | 1.76E−09 | 0.05082467 | −0.01916494 | 9.94E−10 |
| 7A-1 | Fruc | 0.01076495 | −0.03756783 | 2.10E−09 | 0.01508831 | −0.05318582 | 2.42E−08 |
| 7A-2 | Fruc | 0.05715811 | −0.04875768 | 2.20E−16 | 0.08401696 | −0.0718274 | 2.20E−16 |
| 1A | sAX | 0.00267105 | −0.000502881 | 2.09E−06 | 0.00299422 | −0.00055269 | 9.23E−08 |
| 1B | sAX | 0.00089993 | −0.000853359 | 0.0002327 | 0.00101574 | −0.00094506 | 1.65E−05 |
| 6B | sAX | 0.0024687 | −0.002622714 | 2.20E−16 | 0.00266693 | −0.00281596 | 2.20E−16 |
| 7A | sAX | 0.00343923 | −0.000829571 | 6.10E−12 | 0.00350951 | −0.00083763 | 2.39E−13 |
| 1A | tAX | 0.0012122 | −0.000217711 | 0.01132 | 0.00220425 | −0.00039579 | 2.43E−05 |
| 1B | tAX | 0.00112402 | −0.001019239 | 2.78E−08 | 0.00139357 | −0.00126376 | 1.36E−09 |
| 2B | tAX | 0.00253859 | −0.003827511 | 2.20E−16 | 0.00210705 | −0.00317723 | 2.20E−16 |
| 6B | tAX | 0.00116966 | −0.00122459 | 6.31E−10 | 0.00160302 | −0.00167836 | 7.03E−14 |
| 7B | tAX | 0.00029988 | −0.000101704 | 0.3603 | 0.00038679 | −0.00013124 | 0.3091 |

In Table 4, “+QTL” indicates the average increase over the population mean of the MAGIC lines carrying this QTL or QTLs, “−QTL”—indicates the average decrease over the population mean of the MAGIC lines NOT carrying this QTL or QTLs.

The improved fructan content of plants having all four QTL markers associated with increased fructan was analyzed for statistical significance and results are shown in Table 6

TABLE 6

| Fructan | Yanco | Narrabri |
|---|---|---|
| “+QTL: 4, 5, 7, 9” | 0.09623891 | 0.1465182 |
| “−QTL: 4, 5, 7, 9” | −0.0901102 | −0.1368083 |
| Significance (p-value) | 3.10E−09 | 5.95E−10 |

The improved soluble arabinoxylan content of plants having all four QTL markers associated with increased soluble arabinoxylan for statistical significance and results are shown in Table 7.

TABLE 7

| sAX | Yanco | Narrabri |
|---|---|---|
| “+QTL: 1, 2, 6, 8” | 0.010995913 | 0.01185609 |
| “−QTL: 1, 2, 6, 8” | −0.005092835 | −0.00549234 |
| Significance (p-value) | 2.74E−07 | 1.16E−07 |

The improved total arabinoxylan content of plants having all five QTL markers associated with increased soluble arabinoxylan for statistical significance and results are shown in Table 8.

TABLE 8

| tAX | Yanco | Narrabri |
|---|---|---|
| “+QTL: 1, 2, 3, 6, 10” | 0.01024498 | 0.01318866 |
| “−QTL: 1, 2, 3, 6, 10” | −0.00721301 | −0.00768475 |
| Significance (p-value) | 1.85E−10 | 2.20E−16 |

The foregoing example demonstrates the statistical significance of the markers and QTLs of the invention, both individually and in combination.

1. A method of identifying a wheat plant that comprising at least one allele of a marker locus wherein the method comprises:
a. obtaining a wheat plant sample; and
b. detecting an increased fructan/arabinoxylan allele of a marker locus located within a chromosomal interval comprising and flanked by a left and right interval marker for QTL 1A, QTL 1B, QTL 2B-1, QTL 2B-2, QTL 2D, QTL 6B, QTL 7A-1, QTL 7A-2, or QTL 7B.

2. The method of claim 1, wherein the method comprises detecting alleles of four distinct marker loci, wherein each of the four marker loci is located within a chromosomal interval comprising and flanked by a left and right interval marker for QTL 1A, QTL 1B, QTL 2B-1, QTL 2B-2, QTL 2D, QTL 6B, QTL 7A-1, QTL 7A-2, or QTL 7B.

3. The method of claim 1, wherein the method comprises detecting alleles of five distinct marker loci, wherein each of the five marker loci is located within the chromosomal interval comprising and flanked by a left and right interval marker for QTL 1A, QTL 1B, QTL 2B-1, QTL 2B-2, QTL 2D, QTL 6B, QTL 7A-1, QTL 7A-2, or QTL 7B, respectively.

4. The method of claim 1, wherein the method further comprises detecting at least one allele of a marker locus located within the chromosomal interval comprising and flanked by a left and right interval marker for QTL 7A-3.

5. The method of claim 1, wherein each left and right interval marker is identified in Table 1.

6. The method of claim 1, wherein the method comprises detecting an allele of at least one SNP selected from the SNPs disclosed in Table 3.

7. A wheat plant identified by the method of claim 1.

8. A method of marker assisted selection comprising:
a. obtaining a first wheat plant having at least one allele of a marker locus for increased fructan/arabinoxylan, wherein the marker locus is located is located within a chromosomal interval comprising and flanked by a left and right interval marker for QTL 1A, QTL 1B, QTL 2B-1, QTL 2B-2, QTL 2D, QTL 6B, QTL 7A-1, QTL 7A-2, or QTL 7B;
b. crossing the first wheat plant to a second wheat plant;
c. evaluating the progeny for the at least one allele; and
d. selecting progeny plants that possess the at least one allele.

9. The method of claim 8, wherein the first wheat plant comprises at least four alleles of four distinct marker loci for increased fructan/arabinoxylan, wherein each of the four marker loci is located within a chromosomal interval comprising and flanked by a left and right interval marker for QTL 1A, QTL 1B, QTL 2B-1, QTL 2B-2, QTL 2D, QTL 6B, QTL 7A-1, QTL 7A-2, or QTL 7B and the method comprises selecting progeny plants that possess the at least four alleles.

10. The method of claim 8, wherein the first wheat plant comprises at least five alleles of five distinct marker loci for increased fructan/arabinoxylan, wherein each of the five marker loci is located within a chromosomal interval comprising and flanked by a left and right interval marker for QTL 1A, QTL 1B, QTL 2B-1, QTL 2B-2, QTL 2D, QTL 6B, QTL 7A-1, QTL 7A-2, or QTL 7B, respectively, and the method comprises selecting progeny plants that possess the at least five alleles.

11. The method of claim 8, wherein the first wheat plant further comprises at least one allele from a marker locus located within the chromosomal interval comprising and flanked by a left and right interval marker for QTL 7A-3.

12. One or more wheat plants selected by the method of claim 8.

13. A wheat crop comprising wheat plants having one or more alleles of marker loci for increased fructan/arabinoxylan flanked by a left and right interval marker for QTL 1A, QTL 1B, QTL 2B-1, QTL 2B-2, QTL 2D, QTL 6B, QTL 7A-1, QTL 7A-2, or QTL 7B.

14. A method of generating flour, the method comprising milling wheat from the crop of claim 13 and thereby producing flour.

15. A method of planting and harvesting a crop of wheat, the method comprising
a. planting a field with wheat seed, wherein at least 80% of the seed planted in the field comprises one or more alleles of a marker locus for increased fructan/arabinoxylan, wherein each of the one or more marker loci is located is located within a chromosomal interval comprising and flanked by a left and right interval marker for QTL 1A, QTL 1B, QTL 2B-1, QTL 2B-2, QTL 2D, QTL 6B, QTL 7A-1, QTL 7A-2, or QTL 7B;
b. growing wheat plants from the planted wheat seed; and
c. harvesting the wheat plants from the field and thereby generating a wheat crop.

16. The method of claim 15, wherein at least 80% of the seed planted in the field comprises at least four alleles of four distinct marker loci for increased fructan/arabinoxylan, wherein each of the distinct loci is flanked by a left and right interval marker for QTL 1A, QTL 1B, QTL 2B-1, QTL 2B-2, QTL 2D, QTL 6B, QTL 7A-1, QTL 7A-2, or QTL 7BQTL 1A, QTL 1B, QTL 2B-1, QTL 2B-2, QTL 2D, QTL 6B, QTL 7A-1, QTL 7A-2, or QTL 7B.

17. The method of claim 15, wherein at least 80% of the seed planted in the field comprises at least five alleles of five distinct marker loci for increased fructan/arabinoxylan, wherein each of the distinct loci is flanked by a left and right interval marker for QTL 1A, QTL 1B, QTL 2B-1, QTL 2B-2, QTL 2D, QTL 6B, QTL 7A-1, QTL 7A-2, or QTL 7BQTL 1A, QTL 1B, QTL 2B-1, QTL 2B-2, QTL 2D, QTL 6B, QTL 7A-1, QTL 7A-2, or QTL 7B.

18. The method of claim 15, wherein at least 90% of the seed planted in the field comprises the alleles of the distinct marker loci.


SEQUENCE LISTING (20 sequences)

SEQ ID NO: 1 - DNA, 19 bp, Artificial Sequence
  IWA3339
atcccttgcgacaaaagck

SEQ ID NO: 2 - DNA, 23 bp, Artificial Sequence
  IWA3536
gggcatttaagacatggtatggk

SEQ ID NO: 3 - DNA, 19 bp, Artificial Sequence
  IWB3175
tggaattcctcctgctccr

SEQ ID NO: 4 - DNA, 20 bp, Artificial Sequence
  IWB3176
tgtcctgcttcttcccagty

SEQ ID NO: 5 - DNA, 23 bp, Artificial Sequence
  IWB52095
ctacattggccatcacacaggay

SEQ ID NO: 6 - DNA, 25 bp, Artificial Sequence
  forward primer for  QTL 1B marker
cggtcattctttcagaaagcatcty

SEQ ID NO: 7 - DNA, 21 bp, Artificial Sequence
  IWB24280
gagtttgacttgatcccgagr

SEQ ID NO: 8 - DNA, 21 bp, Artificial Sequence
  IWB28342
cacgcttcatgtttttctccr

SEQ ID NO: 9 - DNA, 21 bp, Artificial Sequence
  IWB27678
cacgcttcatgtttttctccy

SEQ ID NO: 10 - DNA, 22 bp, Artificial Sequence
  IWA2961
gtcacatcgtttattaaccgcr

SEQ ID NO: 11 - DNA, 25 bp, Artificial Sequence
  IWB38811
tgtcgcacacctagttgtctgtaay

SEQ ID NO: 12 - DNA, 18 bp, Artificial Sequence
  IWA6420
agcagtctccacgtagcy

SEQ ID NO: 13 - DNA, 20 bp, Artificial Sequence
  IWA44281
gaagatcccaccacttgacy

SEQ ID NO: 14 - DNA, 18 bp, Artificial Sequence
  IWA21840
tgaacggaagctgctccy

SEQ ID NO: 15 - DNA, 19 bp, Artificial Sequence
  IWA56709
acaatcaccgctggcttcy

SEQ ID NO: 16 - DNA, 23 bp, Artificial Sequence
  IWA8231
aatggtttttgtgtgagttctgr

SEQ ID NO: 17 - DNA, 18 bp, Artificial Sequence
  IWB11397
gcaccgtcagcaaggacy

SEQ ID NO: 18 - DNA, 18 bp, Artificial Sequence
  reverse primer QTL 7A-3 marker
gcaccgtcagcaaggacr

SEQ ID NO: 19 - DNA, 25 bp, Artificial Sequence
  IWB72227
gtttgtttgatcctgttaaggctak

SEQ ID NO: 20 - DNA, 24 bp, Artificial Sequence
  IWB34191
cacctctaggatggaaatagcaar